(19) United States
(12) Patent Application Publication
(10) Pub. No.: US 2018/0125834 A1
(43) Pub. Date: May 10, 2018

(54) METHODS OF TREATING BACTERIAL INFECTIONS AND FUNGAL INFECTIONS USING ENANTIOPURE DEUTERIUM-ENRICHED PIOGLITAZONE

(71) Applicant: DeuteRx, LLC, Andover, MA (US)

(72) Inventors: Sheila DeWitt, Auburn, NH (US); Vincent Jacques, Somerville, MA (US); Leonardus H.T. van der Ploeg, Newton, MA (US)

(21) Appl. No.: 15/705,547

(22) Filed: Sep. 15, 2017

Related U.S. Application Data
(63) Continuation of application No. PCT/US2016/023011, filed on Mar. 18, 2016.
(60) Provisional application No. 62/136,005, filed on Mar. 20, 2015.

(51) Int. Cl. A61K 31/4439; A61P 31/04; A61P 31/00

(52) U.S. Cl. CPC A61K 31/4439; A61P 31/00; A61P 31/04

(57) ABSTRACT

The invention provides enantiopure deuterium-enriched pioglitazone, pharmaceutical compositions, and methods of treating bacterial infections and fungal infections using enantiopure deuterium-enriched pioglitazone.

### CROSS-REFERENCE TO RELATED APPLICATIONS

This application claims the benefit of and priority to U.S. Provisional Patent Application Ser. No. 62/136,005, filed Mar. 20, 2015, the contents of which are hereby incorporated by reference.

### FIELD OF THE INVENTION

The invention provides enantiopure deuterium-enriched pioglitazone, pharmaceutical compositions, and methods of treating bacterial infections and fungal infections using enantiopure deuterium-enriched pioglitazone.

### BACKGROUND

Infections by bacteria and fungus affect a substantial number of humans, animals, and plants. Frequently observed bacterial infections in human patients include infections by Staphylococcus species, Mycobacterium tuberculosis, and Escherichia coli. Use of antibiotics for many years to treat these and other bacterial infections is leading to increased frequency of bacterial infections resistant to currently available therapies. Exemplary bacteria reported to have developed resistance to certain antibiotics include Mycobacterium Tuberculosis and Escherichia coli, along certain types of Klebsiella, Clostridium, and Streptococcus bacteria. Certain reports even indicate that up to approximately one half of bacterial infections diagnosed in the United States are caused by resistant strains of bacteria. A particularly acute need exists for new treatments for methicillin-resistant Staphylococcus aureus, beta-lactam-resistant Streptococcus pneumoniae, vancomycin-resistant Enterococci bacteria, and drug-resistant Pseudomonas aeruginosa.

Fungal infections afflict not only human and animal patients but also plants, including plants used in agriculture. Similar to the drug-resistance observed for bacteria, use of agents to treat fungal infections has caused some fungus, such as Candida albicans, Candida glabrata, and Aspergillus spp., to develop resistance to currently available treatments for fungal infections. Patients having a weakened immune system are particularly vulnerable to serious illness associated with fungal infections resistant to current treatment methods.

Accordingly, the need exists for new therapeutic agents and treatment approaches for bacterial infections and fungal infections. The present invention addresses this need and provides other related advantages.

### SUMMARY OF THE INVENTION

The invention provides enantiopure deuterium-enriched pioglitazone, pharmaceutical compositions, and methods of treating bacterial infections and fungal infections using the enantiopure deuterium-enriched pioglitazone. The deuterated pioglitazone contains deuterium enrichment at the chiral center of pioglitazone and optionally in other locations in the compound. Further, the deuterium-enriched pioglitazone is provided in enantiomerically pure form. This enantiomerically pure, deuterium-enriched pioglitazone provides for a better therapeutic agent than non-deuterated pioglitazone and/or racemic mixtures of deuterium-enriched pioglitazone.

Accordingly, one aspect of the invention provides a deuterium-enriched compound of Formula I for use in the therapeutic methods and pharmaceutical compositions described herein. Desirably, the deuterium-enriched compound of Formula I has an optical purity of at least 75% enantiomeric excess. Formula I is represented by:

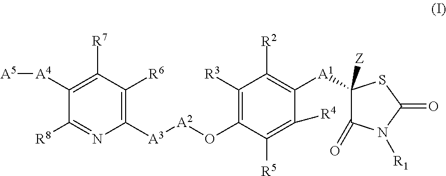

or a pharmaceutically acceptable salt thereof, wherein:

A¹, A², A³, and A⁴ are independently —C(R⁹)(R¹⁰)—;

A⁵ is —C(R¹¹)(R¹²)(R¹³);

R¹, R², R³, R⁴, R⁵, R⁶, R⁷, and R⁸ are independently H or D;

R⁹, R¹⁰, R¹¹, R¹², and R¹³ each represent independently for each occurrence H or D; and

Z is H or D, provided that the abundance of deuterium in Z is at least 30%.

In certain embodiments, the deuterium-enriched compound used in the therapeutic methods and pharmaceutical compositions has the following structure:

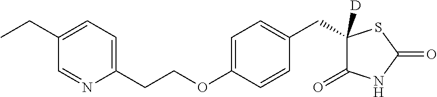 [SMILES: [2H][C@@]1(Cc2ccc(OCCc3ccc(CC)cn3)cc2)SC(=O)NC1=O]

or is a pharmaceutically acceptable salt thereof, each having an optical purity of at least 90% enantiomeric excess.

Another aspect of the invention provides a deuterium-enriched compound of Formula II for use in the therapeutic methods and pharmaceutical compositions described herein. Desirably, the deuterium-enriched compound of Formula II has an optical purity of at least 75% enantiomeric excess. Formula II is represented by:

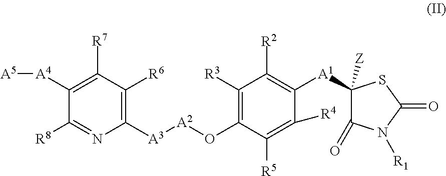

or a pharmaceutically acceptable salt thereof, wherein:

A¹, A², A³, and A⁴ are independently —C(R⁹)(R¹⁰)—,

A⁵ is —C(R¹¹)(R¹²)(R¹³);

R¹, R², R³, R⁴, R⁵, R⁶, R⁷, and R⁸ are independently H or D;

R⁹, R¹⁰, R¹¹, R¹², and R¹³ each represent independently for each occurrence H or D; and

Z is H or D, provided that the abundance of deuterium in Z is at least 30%.

In certain embodiments, the deuterium-enriched compound used in the therapeutic methods and pharmaceutical compositions has the following structure:

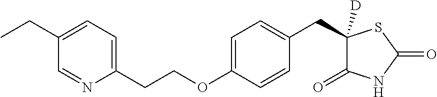 [SMILES: [2H][C@]1(Cc2ccc(OCCc3ccc(CC)cn3)cc2)SC(=O)NC1=O]

or is a pharmaceutically acceptable salt thereof, each having an optical purity of at least 90% enantiomeric excess.

The deuterium-enriched compounds are particularly useful in the treatment of bacterial infections and fungal infections. Exemplary bacterial infections include, for example, an infection by Streptococcus pneumonia, Escherichia coli, and/or Klebsiella pneumoniae. Exemplary fungal infections include, for example, an infection by a member of the genus Acremonium, Alternaria, Aspergillus, Basidiobolus, Penicillium, Sporothrix, and/or Trichophyton.

Accordingly, one aspect of the invention provides a method of treating an infection selected from the group consisting of a bacterial infection and a fungal infection. The method comprises administering to a patient in need thereof a therapeutically effective amount of a deuterium-enriched compound described herein, such as a compound of Formula I or Formula II, having an optical purity of at least 75% enantiomeric excess to treat the infection. In certain embodiments, the deuterium-enriched compound is a compound of Formula I. In certain other embodiments, the deuterium-enriched compound is a compound of Formula II.

### BRIEF DESCRIPTION OF THE DRAWINGS

FIG. 1 is a line graph depicting results of PPARγ agonist activity testing for d-S-pio, d-R-pio, and h-rac-pio, as further described in Example 3.

FIG. 2A is a line graph depicting in vitro stability data for h-S-pio in human plasma in the form of experimental data points and results from fitting to kinetic differential equations, as further described in Example 4. The abbreviation “calc” indicates results from fitting experimental data to kinetic differential equations.

FIG. 2B is a line graph depicting in vitro stability data for h-R-pio in human plasma in the form of experimental data points and results from fitting to kinetic differential equations, as further described in Example 4. The abbreviation “calc” indicates results from fitting experimental data to kinetic differential equations.

FIG. 2C is a line graph depicting in vitro stability data for d-rac-pio in human plasma in the form of experimental data points and results from fitting to kinetic differential equations, as further described in Example 4. The abbreviation “calc” indicates results from fitting experimental data to kinetic differential equations.

FIG. 3A is a line graph depicting in vitro stability data for h-S-pio in mouse plasma in the form of experimental data points and results from fitting to kinetic differential equations, as further described in Example 4. The abbreviation “calc” indicates results from fitting experimental data to kinetic differential equations.

FIG. 3B is a line graph depicting in vitro stability data for h-R-pio in mouse plasma in the form of experimental data points and results from fitting to kinetic differential equations, as further described in Example 4. The abbreviation “calc” indicates results from fitting experimental data to kinetic differential equations.

FIG. 3C is a line graph depicting in vitro stability data for d-rac-pio in mouse plasma in the form of experimental data points and results from fitting to kinetic differential equations, as further described in Example 4. The abbreviation “calc” indicates results from fitting experimental data to kinetic differential equations.

FIG. 4 is a bar graph depicting maximal respiration as oxygen consumption rate (OCR in pmoles O₂/min) of C2C12 cells treated with h-rac-pio, d-S-pio, or d-R-pio at 30 μM for 15, 30, or 90 min compared to the OCR in vehicle-treated cells, as further described in Example 6.

FIG. 5A is a line graph depicting PK profiles for the enantiomers of pioglitazone in mice administered h-rac-pio (30 mg/kg) by oral gavage daily for 5 days; (S)-enantiomer—hollow triangles, dashed line; (R)-enantiomer—hollow squares, dotted line; as further described in Example 7.

FIG. 5B is a line graph depicting PK profiles for the enantiomers of pioglitazone in mice administered d-R-pio (15 mg/kg) by oral gavage daily for 5 days; (S)-enantiomer—hollow triangles, dashed line; (R)-enantiomer—hollow squares, dotted line; each curve represents the sum of corresponding isotopomers ((S)-enantiomer: h-S-pio+d-S-pio, and (R)-enantiomer: h-R-pio+d-R-pio), as further described in Example 7.

FIG. 5C is a line graph depicting PK profiles for the enantiomers of pioglitazone in mice administered d-S-pio (15 mg/kg) by oral gavage daily for 5 days; (S)-enantiomer—hollow triangles, dashed line; (R)-enantiomer—hollow squares, dotted line; each curve represents the sum of corresponding isotopomers ((S)-enantiomer: h-S-pio+d-S-pio, and (R)-enantiomer: h-R-pio+d-R-pio), as further described in Example 7.

### DETAILED DESCRIPTION

The invention provides enantiopure deuterium-enriched pioglitazone, pharmaceutical compositions, and methods of treating bacterial infections and fungal infections using enantiopure deuterium-enriched pioglitazone. Deuterium-enriched refers to the feature that the compound has a quantity of deuterium that is greater than in naturally occurring compounds or synthetic compounds prepared from substrates having the naturally occurring distribution of isotopes. The threshold amount of deuterium enrichment is specified in certain instances in this disclosure, and all percentages given for the amount of deuterium present are mole percentages.

Deuterium (²H) is a stable, non-radioactive isotope of ¹H hydrogen and has an atomic weight of 2.014. Hydrogen naturally occurs as a mixture of the isotopes ¹H hydrogen (i.e., protium), deuterium (²H), and tritium (³H). The natural abundance of deuterium is 0.015%. One of ordinary skill in the art recognizes that in all chemical compounds with an H atom, the H atom actually represents a mixture of ¹H hydrogen, deuterium (²H), and tritium (³H), where about 0.015% is deuterium. Thus, compounds with a level of deuterium that has been enriched to be greater than its natural abundance of 0.015% are considered unnatural and, as a result, novel over their non-enriched counterparts.

The deuterium-enriched pioglitazone described herein contains deuterium enrichment at the chiral center of pioglitazone and optionally in other locations in the compound. Deuterium-enrichment at the chiral center reduces the rate at which the two enantiomers of pioglitazone may interconvert. Further, the deuterium-enriched pioglitazone described herein is provided in enantiomerically pure form. This enantiomerically pure, deuterium-enriched pioglitazone provides for a better therapeutic agent than non-deuterated pioglitazone and/or racemic mixtures of the compound.

Exemplary compositions and methods of the present invention are described in more detail in the following sections: I. Deuterium-enriched Pioglitazone; II. Therapeutic Applications; III. Dosing Considerations and Combination Therapy, and IV. Pharmaceutical Compositions. Aspects of the invention described in one particular section are not to be limited to any particular section.

### I. Deuterium-Enriched Pioglitazone

One aspect of the invention provides deuterium-enriched compounds for use in the therapeutic methods and pharmaceutical compositions described herein. The deuterium-enriched compounds are provided in high enantiomeric purity in order to maximize therapeutic benefit, such as maximal potency per dose of therapeutic agent and minimize adverse side effects.

One such deuterium-enriched compound is a family of deuterium-enriched compounds represented by Formula I having an optical purity of at least 75% enantiomeric excess:

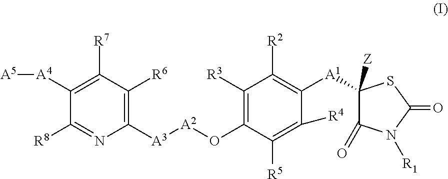

or a pharmaceutically acceptable salt thereof, wherein:

A¹, A², A³, and A⁴ are independently —C(R⁹)(R¹⁰)—;

A⁵ is —C(R¹¹)(R¹²)(R¹³);

R¹, R², R³, R⁴, R⁵, R⁶, R⁷, and R⁸ are independently H or D;

R⁹, R¹⁰, R¹¹, R¹², and R¹³ each represent independently for each occurrence H or D; and

Z is H or D, provided that the abundance of deuterium in Z is at least 30%.

In certain embodiments, A¹ is —CH₂—. In certain embodiments, A² is —CH₂—. In certain embodiments, A³ is —CH₂—. In certain embodiments, A⁴ is —CH₂—. In certain embodiments, A² and A³ are —CH₂—. In certain other embodiments, A¹, A², A³, and A⁴ are —CH₂—.

In certain embodiments, A⁵ is —CH₃. In certain embodiments, A⁴ is —CH₂—, and A⁵ is —CH₃.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, and R⁸ are H.

The description above describes multiple embodiments relating to compounds of Formula I. The patent application specifically contemplates all combinations of the embodiments. For example, the invention contemplates a compound of Formula I wherein R¹, R², R³, R⁴, R⁵, R⁶, R⁷, and R⁸ are H; A¹, A², A³, and A⁴ are —CH₂—; and A⁵ is —CH₃.

Another such deuterium-enriched compound is a family of deuterium-enriched compounds represented by Formula I-A having an optical purity of at least 75% enantiomeric excess:

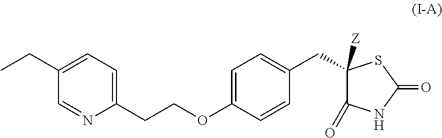

or a pharmaceutically acceptable salt thereof, wherein Z is H or D, provided that the abundance of deuterium in Z is at least 30%.

The compounds of Formula I and Formula I-A can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (0 at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I and Formula I-A can be further characterized according their enantiomeric purity. In certain embodiments, the deuterium-enriched compound has an enantiomeric excess of at least 80%, 85%, 90%, 95%, or 98%. Still further examples of the optical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 1 and 2 below.

TABLE 1

| Compound | |
|---|---|
| No. | Structure |
| 1 | 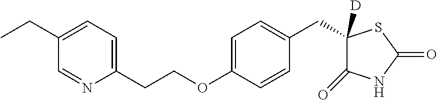 [SMILES: [2H][C@@]1(Cc2ccc(OCCc3ccc(CC)cn3)cc2)SC(=O)NC1=O] |
| | or a pharmaceutically |
| | acceptable salt thereof, each having an optical purity of at least 60% |
| | enantiomeric excess. |
| 2 | 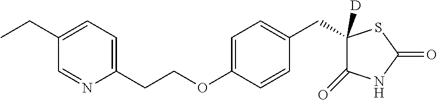 [SMILES: [2H][C@@]1(Cc2ccc(OCCc3ccc(CC)cn3)cc2)SC(=O)NC1=O] |
| | having an optical purity of |
| | at least 60% enantiomeric excess. |
| 3 | 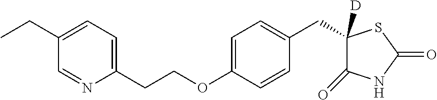 [SMILES: [2H][C@@]1(Cc2ccc(OCCc3ccc(CC)cn3)cc2)SC(=O)NC1=O] |
| | hydrochloride having an |
| | optical purity of at least 60% enantiomeric excess. |
| 4 | 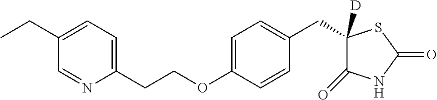 [SMILES: [2H][C@@]1(Cc2ccc(OCCc3ccc(CC)cn3)cc2)SC(=O)NC1=O] |
| | or a pharmaceutically |
| | acceptable salt thereof, each having an optical purity of at least 75% |
| | enatiomeric excess. |
| 5 | 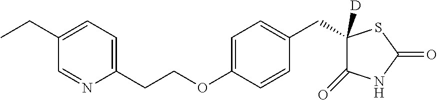 [SMILES: [2H][C@@]1(Cc2ccc(OCCc3ccc(CC)cn3)cc2)SC(=O)NC1=O] |
| | having an optical purity of |
| | at least 75% enantiomeric excess. |
| 6 | 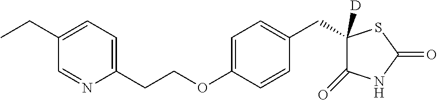 [SMILES: [2H][C@@]1(Cc2ccc(OCCc3ccc(CC)cn3)cc2)SC(=O)NC1=O] |
| | hydrochloride having an |
| | optical purity of at least 75% enantiomeric excess. |
| 7 | 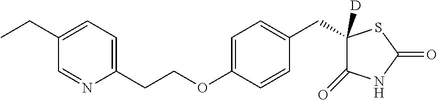 [SMILES: [2H][C@@]1(Cc2ccc(OCCc3ccc(CC)cn3)cc2)SC(=O)NC1=O] |
| | or a pharmaceutically |
| | acceptable salt thereof, each having an optical purity of at least 90% |
| | enantiomeric excess. |
| 8 | 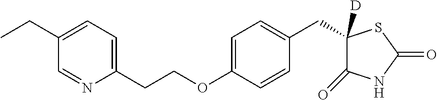 [SMILES: [2H][C@@]1(Cc2ccc(OCCc3ccc(CC)cn3)cc2)SC(=O)NC1=O] |
| | having an optical purity of |
| | at least 90% enantiomeric excess. |
| 9 | 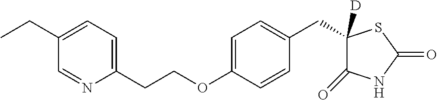 [SMILES: [2H][C@@]1(Cc2ccc(OCCc3ccc(CC)cn3)cc2)SC(=O)NC1=O] |
| | hydrochloride having an |
| | optical purity of at least 90% enantiomeric excess. |
| 10 | 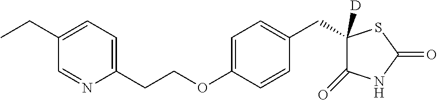 [SMILES: [2H][C@@]1(Cc2ccc(OCCc3ccc(CC)cn3)cc2)SC(=O)NC1=O] |
| | or a pharmaceutically |
| | acceptable salt thereof, each having an optical purity of at least 95% |
| | enantiomeric excess. |
| 11 | 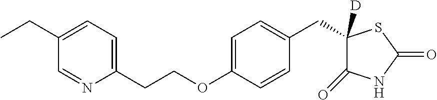 [SMILES: [2H][C@@]1(Cc2ccc(OCCc3ccc(CC)cn3)cc2)SC(=O)NC1=O] |
| | having an optical purity of |
| | at least 95% enantiomeric excess. |
| 12 | 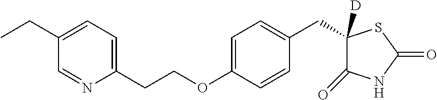 [SMILES: [2H][C@@]1(Cc2ccc(OCCc3ccc(CC)cn3)cc2)SC(=O)NC1=O] |
| | hydrochloride having an |
| | optical purity of at least 95% enantiomeric excess. |

TABLE 2

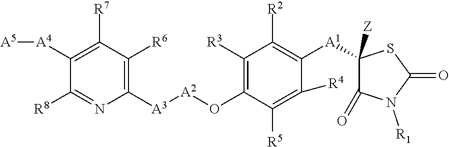

| Com- | |
|---|---|
| pound | |
| No. | Variable Definition |
| 1 | Z = D; R¹ = D; R²-R⁸ are H; A¹, A², A³, and A⁴ are —CH₂—; and A⁵ is —CH₃ |
| 2 | Z = D; R¹-R⁸ are H; A¹ = —CD₂—; A², A³, and A⁴ are —CH₂—; and A⁵ is —CH₃ |
| 3 | Z = D; R¹ = H; R², R³, R⁴, and R⁵ are D; R⁶-R⁸ are H; A¹, A², A³, and A⁴ are —CH₂—; |
| | and A⁵ is —CH₃ |
| 4 | Z = D; R¹-R⁸ are H; A¹ = —CH₂—; A² and A³ are —CD₂—; A⁴ = —CH₂—; and A⁵ is —CH₃ |
| 5 | Z = D; R¹, R², R³, R⁴, and R⁵ are H; R⁶-R⁸ are D; A¹, A², A³, and A⁴ are —CH₂—; and |
| | A⁵ is —CH₃ |
| 6 | Z = D; R¹-R⁸ are H; A¹, A², and A³ are —CH₂—; A⁴ are —CD₂—; and A⁵ is —CD₃ |

Another embodiment of the invention provides a compound in Table 2 wherein the compound has an enantiomeric excess of at least 60%, 70%, 75%, 80%, 85%, 90%, 95%, or 98%.

Another such deuterium-enriched compound is a family of deuterium-enriched compounds represented by Formula II having an optical purity of at least 75% enantiomeric excess:

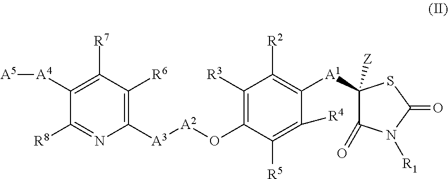

or a pharmaceutically acceptable salt thereof, wherein:

A¹, A², A³, and A⁴ are independently —C(R⁹)(R¹⁰)—,

A⁵ is —C(R¹¹)(R¹²)(R¹³);

R¹, R², R³, R⁴, R⁵, R⁶, R⁷, and R⁸ are independently H or D;

R⁹, R¹⁰, R¹¹, R¹², and R¹³ each represent independently for each occurrence H or D; and

Z is H or D, provided that the abundance of deuterium in Z is at least 30%.

In certain embodiments, A¹ is —CH₂—. In certain embodiments, A² is —CH₂—. In certain embodiments, A³ is —CH₂—. In certain embodiments, A⁴ is —CH₂—. In certain embodiments, A² and A³ are —CH₂—. In certain other embodiments, A¹, A², A³, and A⁴ are —CH₂—.

In certain embodiments, A⁵ is —CH₃. In certain embodiments, A⁴ is —CH₂—, and A⁵ is —CH₃.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, and R⁸ are H.

The description above describes multiple embodiments relating to compounds of Formula II. The patent application specifically contemplates all combinations of the embodiments. For example, the invention contemplates a compound of Formula II wherein R¹, R², R³, R⁴, R⁵, R⁶, R⁷, and R⁸ are H; A¹, A², A³, and A⁴ are —CH₂—; and A⁵ is —CH₃.

Another such deuterium-enriched compound is a family of deuterium-enriched compounds represented by Formula II-A having an optical purity of at least 75% enantiomeric excess:

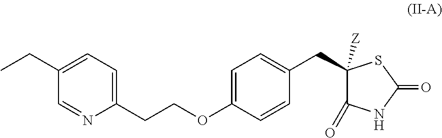

or a pharmaceutically acceptable salt thereof, wherein Z is H or D, provided that the abundance of deuterium in Z is at least 30%.

The compounds of Formula II and Formula II-A can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (0 at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II and Formula II-A can be further characterized according their enantiomeric purity. In certain embodiments, the deuterium-enriched compound has an enantiomeric excess of at least 80%, 85%, 90%, 95%, or 98%. Still further examples of the optical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 3 and 4 below.

TABLE 3

| Compound | |
|---|---|
| No. | Structure |
| 1 | 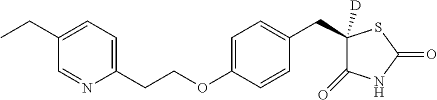 [SMILES: [2H][C@]1(Cc2ccc(OCCc3ccc(CC)cn3)cc2)SC(=O)NC1=O] |
| | or a pharmaceutically |
| | acceptable salt thereof, each having an optical purity of at least 60% |
| | enantiomeric excess. |
| 2 | 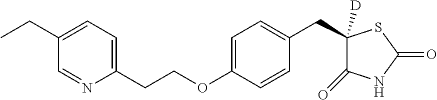 [SMILES: [2H][C@]1(Cc2ccc(OCCc3ccc(CC)cn3)cc2)SC(=O)NC1=O] |
| | having an optical purity of |
| | at least 60% enantiomeric excess. |
| 3 | 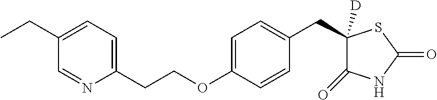 [SMILES: [2H][C@]1(Cc2ccc(OCCc3ccc(CC)cn3)cc2)SC(=O)NC1=O] |
| | hydrochloride having an |
| | optical purity of at least 60% enantiomeric excess. |
| 4 | 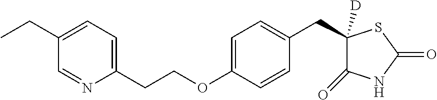 [SMILES: [2H][C@]1(Cc2ccc(OCCc3ccc(CC)cn3)cc2)SC(=O)NC1=O] |
| | or a pharmaceutically |
| | acceptable salt thereof, each having an optical purity of at least 75% |
| | enantiomeric excess. |
| 5 | 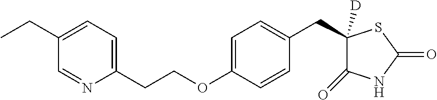 [SMILES: [2H][C@]1(Cc2ccc(OCCc3ccc(CC)cn3)cc2)SC(=O)NC1=O] |
| | having an optical purity of |
| | at least 75% enantiomeric excess. |
| 6 | 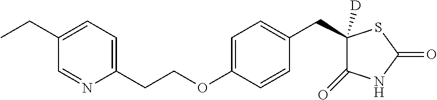 [SMILES: [2H][C@]1(Cc2ccc(OCCc3ccc(CC)cn3)cc2)SC(=O)NC1=O] |
| | hydrochloride having an |
| | optical purity of at least 75% enantiomeric excess. |
| 7 | 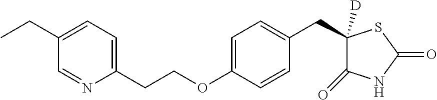 [SMILES: [2H][C@]1(Cc2ccc(OCCc3ccc(CC)cn3)cc2)SC(=O)NC1=O] |
| | or a pharmaceutically |
| | acceptable salt thereof, each having an optical purity of at least 90% |
| | enantiomeric excess. |
| 8 | 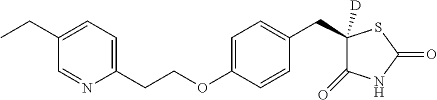 [SMILES: [2H][C@]1(Cc2ccc(OCCc3ccc(CC)cn3)cc2)SC(=O)NC1=O] |
| | having an optical purity of |
| | at least 90% enantiomeric excess. |
| 9 | 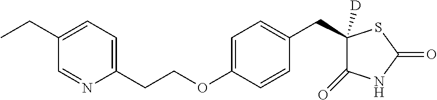 [SMILES: [2H][C@]1(Cc2ccc(OCCc3ccc(CC)cn3)cc2)SC(=O)NC1=O] |
| | hydrochloride having an |
| | optical purity of at least 90% enantiomeric excess. |
| 10 | 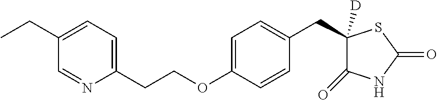 [SMILES: [2H][C@]1(Cc2ccc(OCCc3ccc(CC)cn3)cc2)SC(=O)NC1=O] |
| | or a pharmacetutically |
| | acceptable salt therof, each having an optical purity of at least 95% |
| | enantiomeric excess. |
| 11 | 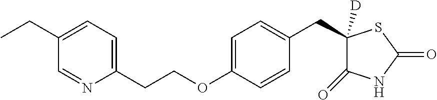 [SMILES: [2H][C@]1(Cc2ccc(OCCc3ccc(CC)cn3)cc2)SC(=O)NC1=O] |
| | having an optical purity of |
| | at least 95% enantiomeric excess. |
| 12 | 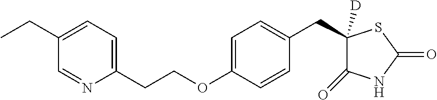 [SMILES: [2H][C@]1(Cc2ccc(OCCc3ccc(CC)cn3)cc2)SC(=O)NC1=O] |
| | hydrochloride having an |
| | optical purity of at least 95% enantiomeric excess. |

TABLE 4

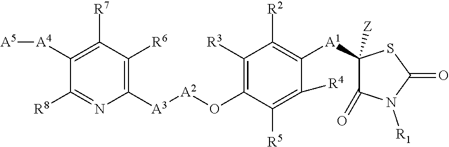

| Com- | |
|---|---|
| pound | |
| No. | Variable Definition |
| 1 | Z = D; R¹ = D; R²-R⁸ are H; A¹, A², A³, and A⁴ are —CH₂—; and A⁵ is —CH₃ |
| 2 | Z = D; R¹-R⁸ are H; A¹ = —CD₂—; A², A³, and A⁴ are —CH₂—; and A⁵ is CH₃ |
| 3 | Z = D; R¹ = H; R², R³, R⁴, and R⁵ are D; R⁶-R⁸ are H; A¹, A², A³, and A⁴ are —CH₂—; |
| | and A⁵ is —CH₃ |
| 4 | Z = D; R¹-R⁸ are H; A¹ = —CH₂—; A² and A³ are —CD₂—; A⁴ = —CH₂—; and A⁵ is —CH₃ |
| 5 | Z = D; R¹, R², R³, R⁴, and R⁵ are H; R⁶-R⁸ are D; A¹, A², A³, and A⁴ are —CH₂—; and |
| | A⁵ is —CH₃ |
| 6 | Z = D; R¹-R⁸ are H; A¹, A², and A³ are —CH₂—; A⁴ are —CD₂—; and A⁵ is —CD₃ |

Another embodiment of the invention provides a compound in Table 4 wherein the compound has an enantiomeric excess of at least 60%, 70%, 75%, 80%, 85%, 90%, 95%, or 98%.

As indicated above, the deuterium-enriched compound may be in the form of a pharmaceutically acceptable salt. One such pharmaceutically acceptable salt is a hydrochloride salt.

It is understood that the deuterium-enriched compounds described herein can be combined with a pharmaceutically acceptable carrier to form a pharmaceutical composition.

Deuterium-enriched compounds of the invention can generally be prepared by substituting a deuterium-enriched reagent for a non-isotopically labeled reagent in synthetic schemes reported in the literature for making non-isotopically labeled pioglitazone. Scheme 1 below illustrates a general method for preparing deuterium-enriched pioglitazone, in which deuterium is incorporated at the sole chiral center. The scheme is provided for the purpose of illustrating the invention, and should not be regarded in any manner as limiting the scope or the spirit of the invention. In Scheme 1, pioglitazone hydrochloride is first stirred with perdeuterated dimethylsulfoxide (d₆-DMSO) and triethylamine and then treated with perdeuterated methanol (d₄-MeOH). The R-enantiomer and S-enantiomer of deutero-thiazolidine A are separated using chiral chromatography, such as chiral high-performance liquid chromatography. Alternatively, the R-enantiomer and S-enantiomer of deutero-thiazolidine A may be separated by reaction with a chiral carboxylic acid to form a salt, followed by separation of the resulting diastereomeric salts, and conversion of the separated salts back to deuterated pioglitazone free base in enantio-pure form. Pioglitazone hydrochloride can be prepared using the methods described in for example, U.S. Pat. No. 4,444,779; EP 193256; U.S. Pat. No. 4,687,777; U.S. Pat. No. 8,173,816; and U.S. Patent Application Publication No. 2011/0021576, each of which is incorporated herein by reference.

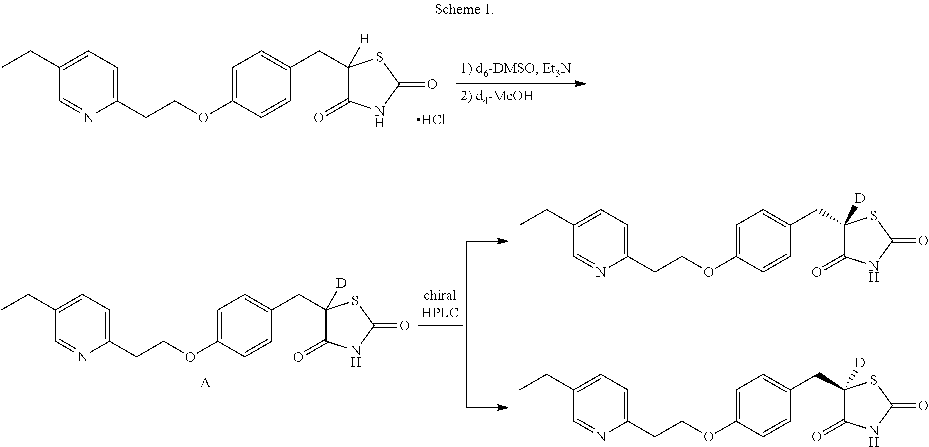

Scheme 2 below illustrates a general method for preparing deuterium-enriched pioglitazone, in which deuterium is incorporated at the ethyl group attached to the pyridine and at the sole chiral center. Reaction of 2-(5-(d₅-ethyl)pyridin-2-yl)ethanol (A1) with p-fluoro-nitrobenzene provides nitrophenyl ether B1. Reduction of nitrophenyl ether B1, such as through hydrogenation in the presence of palladium/carbon, provides aminophenyl ether C1. Reaction of aminophenyl ether C1 with NaNO₂ and hydrobromic acid, followed by addition of CH₂═CHCO₂Et provides alpha-bromo ester D1. Reaction of alpha-bromo ester D1 with thiourea provides thiazolidine-2,4-dione E1. Reaction of thiazolidine-2,4-dione E1 with d₆-DMSO and triethylamine, followed by d₄-MeOH provides deutero-thiazolidine-2,4-dione F1. The R-enantiomer and S-enantiomer of deutero-thiazolidine-2,4-dione F1 are separated using chiral chromatography, such as chiral high-performance liquid chromatography.

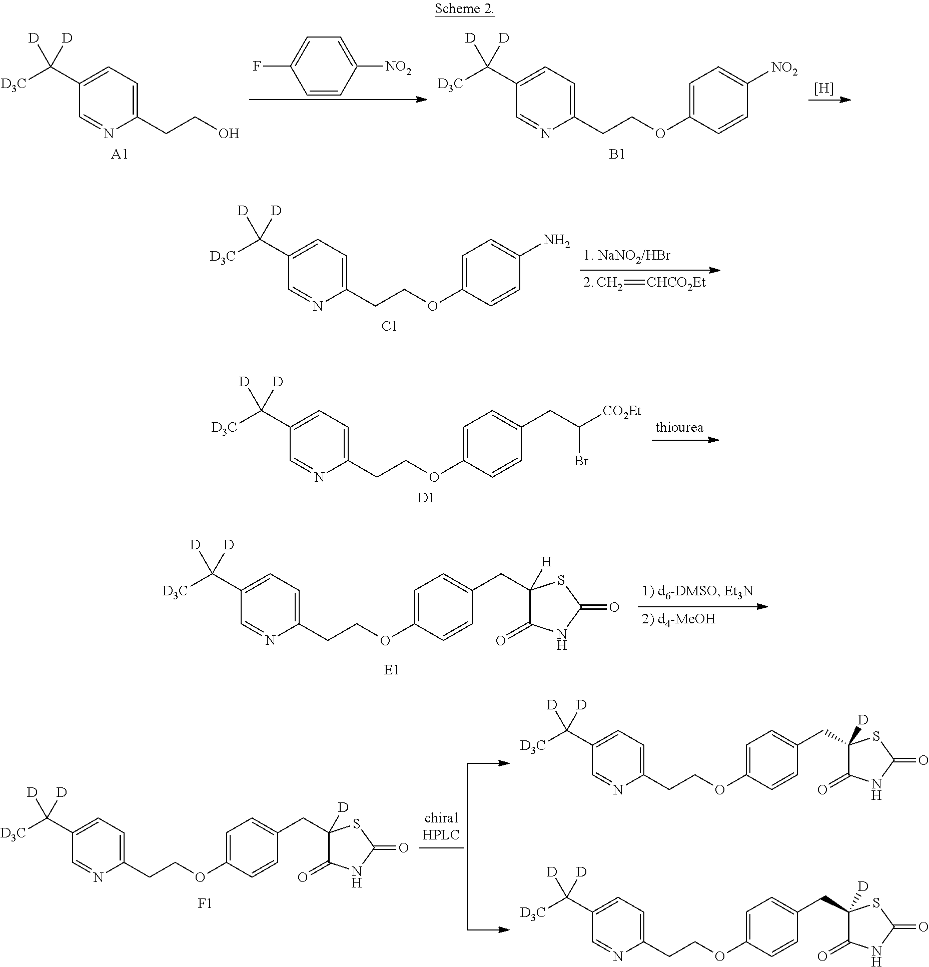

Compounds having deuterium enrichment at a position other than the ethyl group on the pyridine can be prepared using other deuterated forms of starting materials shown in Scheme 2 (e.g., a deuterated form of p-fluoro-nitrobenzene).

Compounds described herein can be provided in isolated or purified form. Isolated or purified compounds are a group of compounds that have been separated from their environment, such as from a crude reaction mixture if made in a laboratory setting or removed from their natural environment if naturally occurring. Examples of the purity of the isolated compound include, for example, at least 50%, 55%, 60%, 65%, 70%, 75%, 80%, 85%, 90%, 95%, to 100% by weight.

Another aspect of the invention provides a unit quantum of a deuterium-enriched compound described herein, such as an amount of at least (a) one μg of a disclosed deuterium-enriched compound, (b) one mg, or (c) one gram. In further embodiments, the quantum is, for example, at least 0.01, 0.02, 0.03, 0.04, 0.05, 0.1, 0.2, 0.3, 0.4, 0.5, or 1 mole of the compound. The present amounts also cover lab-scale (e.g., gram scale including 1, 2, 3, 4, 5 g, etc.), kilo-lab scale (e.g., kilogram scale including 1, 2, 3, 4, 5 kg, etc.), and industrial or commercial scale (e.g., multi-kilogram or above scale including 100, 200, 300, 400, 500 kg, etc.) quantities as these will be more useful in the actual manufacture of a pharmaceutical. Industrial/commercial scale refers to the amount of product that would be produced in a batch that was designed for clinical testing, formulation, sale/distribution to the public, etc.

### II. Therapeutic Applications

The invention provides methods of using deuterium-enriched compounds described herein to treat bacterial infections and fungal infections. Use of the deuterium-enriched compounds having high enantiomeric purity is contemplated to maximize therapeutic benefit, such as achieving increased potency per dose of therapeutic agent and minimize adverse side effects. The bacterial infection and fungal infection may be afflicting a patient, such as a human or veterinary animal, or may be afflicting a plant, such as an agricultural crop.

Accordingly, one aspect of the invention provides a method of treating an infection selected from the group consisting of a bacterial infection and a fungal infection. The method comprises administering to a patient in need thereof a therapeutically effective amount of a deuterium-enriched compound described herein, such as a compound of Formula I or Formula II, having an optical purity of at least 75% enantiomeric excess to treat the infection. The deuterium-enriched compound can be, for example, a compound of Formula I, Formula I-A, Formula II, Formula II-A, or one of the other deuterium-enriched compounds described in Section I above. In certain embodiments, the infection is a bacterial infection. In certain other embodiments, the infection is a fungal infection. Exemplary bacterial infections and fungal infections contemplated for treatment are described in more detail below.

Another aspect of the invention provides a method of treating an infection selected from the group consisting of a bacterial infection and a fungal infection. The method comprises exposing a bacteria or fungus to an effective amount of a deuterium-enriched compound described herein, such as a compound of Formula I or Formula II, having an optical purity of at least 75% enantiomeric excess to treat the infection. The deuterium-enriched compound can be, for example, a compound of Formula I, Formula I-A, Formula II, Formula II-A, or one of the other deuterium-enriched compounds described in Section I above. In certain embodiments, the infection is a bacterial infection. In certain other embodiments, the infection is a fungal infection. Exemplary bacterial infections and fungal infections contemplated for treatment are described below.

Another aspect of the invention provides a method of inducing death of a bacterial cell. The method comprises exposing a bacterial cell to an effective amount of a deuterium-enriched compound described herein, such as a compound of Formula I or Formula II, having an optical purity of at least 75% enantiomeric excess to induce death of said bacterial cell. The deuterium-enriched compound can be, for example, a compound of Formula I, Formula I-A, Formula II, Formula II-A, or one of the other deuterium-enriched compounds described in Section I above. In certain embodiments, the method comprises inducing death of a population of bacterial cells.

Another aspect of the invention provides a method of inducing death of a fungus. The method comprises exposing a fungus to an effective amount of a deuterium-enriched compound described herein, such as a compound of Formula I or Formula II, having an optical purity of at least 75% enantiomeric excess to induce death of said fungus. The deuterium-enriched compound can be, for example, a compound of Formula I, Formula I-A, Formula II, Formula II-A, or one of the other deuterium-enriched compounds described in Section I above. In certain embodiments, the method comprises inducing death of a population of fungus.

### Bacteria for Treatment/Induction of Cell Death

Bacteria can be characterized according to classifications known in the art. For example, in certain embodiments, the bacteria is a gram-positive bacteria, such as a gram-positive coccus bacteria or a gram-positive bacillus bacteria. In other embodiments, the bacteria is a gram-negative bacteria, such as a gram-negative coccus bacteria or a gram-negative bacillus bacteria. The bacteria can also be characterized according to whether it is an anaerobic or aerobic bacteria. Accordingly, in certain embodiments, the bacteria is an anaerobic bacteria. In certain other embodiments, the bacteria is an aerobic bacteria.

A variety of bacteria are contemplated to be susceptible to the deuterium-enriched compounds herein. Representative bacteria include Staphylococci species, e.g., S. aureus; Enterococci species, e.g., E. faecalis and E. faecium; Streptococci species, e.g., S. pyogenes and S. pneumoniae; Escherichia species, e.g., E. coli, including enterotoxigenic, enteropathogenic, enteroinvasive, enterohemorrhagic and enteroaggregative E. coli strains; Haemophilus species, e.g., H. influenza; and Moraxella species, e.g., M. catarrhalis. Other examples include Mycobacteria species, e.g., M. tuberculosis, M. avian-intracellulare, M. kansasii, M. bovis, M. africanum, M. genavense, M. leprae, M. xenopi, M. simiae, M. scrofulaceum, M. malmoense, M. celatum, M. abscessus, M. chelonae, M. szulgai, M. gordonae, M. haemophilum, M. fortuni and M. marinum; Corynebacteria species, e.g., C. diphtheriae; Vibrio species, e.g., V. cholerae; Campylobacter species, e.g., C. jejuni; Helicobacter species, e.g., H. pylori; Pseudomonas species, e.g., P. aeruginosa; Legionella species, e.g., L. pneumophila; Treponema species, e.g., T. pallidum; Borrelia species, e.g., B. burgdorferi; Listeria species, e.g., L. monocytogenes; Bacillus species, e.g., B. cereus; Bordatella species, e.g., B. pertussis; Clostridium species, e.g., C. perfringens, C. tetani, C. difficile and C. botulinum; Neisseria species, e.g., N. meningitidis and N. gonorrhoeae; Chlamydia species, e.g., C. psittaci, C. pneumoniae and C. trachomatis; Rickettsia species, e.g., R. rickettsii and R. prowazekii; Shigella species, e.g., S. sonnei; Salmonella species, e.g., S. typhimurium; Yersinia species, e.g., Y. enterocolitica and Y. pseudotuberculosis; Klebsiella species, e.g., K. pneumoniae; Mycoplasma species, e.g., M. pneumoniae; and Trypanosoma brucei. In certain embodiments, the compounds described herein are used to treat a patient suffering from a bacterial infection selected from the group consisting of S. aureus, E. faecalis, E. faecium, S. pyogenes, S. pneumonia, and P. aeruginosa.

In yet other embodiments, the bacteria is a member of the genus Peptostreptococci, a Peptostreptococci asaccharolyticus, a Peptostreptococci magnus, a Peptostreptococci micros, a Peptostreptococci prevotii, a member of the genus Porphyromonas, a Porphyromonas asaccharolytica, a Porphyromonas canoris, a Porphyromonas gingivalis, a Porphyromonas macaccae, a member of the genus Actinomyces, an Actinomyces israelii, an Actinomyces odontolyticus, a member of the genus Clostridium, a Clostridium innocuum, a Clostridium clostridioforme, a Clostridium difficile, a member of the genus Anaerobiospirillum, a member of the genus Bacteroides, a Bacteroides tectum, a Bacteroides ureolyticus, a Bacteroides gracilis (Campylobacter gracilis), a member of the genus a Prevotella, a Prevotella intermedia, a Prevotella heparinolytica, a Prevotella oris-buccae, a Prevotella bivia, a Prevotella melaninogenica, a member of the genus Fusobacterium, a Fusobacterium naviforme, a Fusobacterium necrophorum, a Fusobacterium varium, a Fusobacterium ulcerans, a Fusobacterium russii, a member of the genus Bilophila, or a Bilophila wadsworthia.

In yet other embodiments, methods herein involve treatment of an infection by one or more of a Streptococccus, Escherichia, Klebsiella, Acinetobacter, Actinomyces, Anaerobiospirillum, Bacillus, Bacteroides, Bilophila, Campylobacter, Clostridium, Enterococcus, Eubacterium, Francisella, Fusobacterium, Haemophilus, Listeria, Moraxella, Mycobacterium, Neisseria, Peptostreptococci, Porphyromonas, Prevotella, Proteus, Pseudomonas, Salmonella, or Yersinia. In certain embodiments, the bacterial infection is an infection by one or more Streptococccus species, Escherichia species, Klebsiella species, Actinomyces species, Enterococcus species, Mycobacterium species, Neisseria species, or Pseudomonas species. In certain other embodiments, the bacterial infection is an infection by one or more of Streptococcus pneumoniae, Streptococcus pyogenes, Staphylococcus aureus, Staphylococcus epidermidis, Acinetobacter baumannii, Bacillus anthraces, Bacteroides fragilis, Clostridium perfringens, Clostridium difficile, Escherichia coli, Enterococcus faecalis, Enterococcus faecium, Eubacterium lentum, Francisella tularensis, Fusobacterium nucleatum, Haemophilus influenzae, Klebsiella pneumoniae, Moraxella catarrhalis, Mycobacterium smegmatis, Mycobacterium tuberculosis, Neisseria gonorrhoeae, Porphyromonas asaccharolyticus, Proteus mirabilis, Pseudomonas aeruginosa, Salmonella typhimurium, or Yersinia enterocolytica. In certain other embodiments, the bacterial infection is an infection by Streptococccus pneumoniae, Escherichia coli, or Klebsiella pneumoniae.

The antibacterial activity of compounds described herein may be evaluated using assays known in the art, such as the microbroth dilution minimum inhibition concentration (MIC) assay, as further described in National Committee for Clinical Laboratory Standards. Performance Standards for Antimicrobial Susceptibility Testing; Fourteenth Informational Supplement. NCCLS document M100-S14 {ISBN 1-56238-516-X}. This assay may be used to determine the minimum concentration of a compound necessary to prevent visible bacterial growth in a solution. In general, the drug to be tested is serially diluted into wells, and aliquots of liquid bacterial culture are added. This mixture is incubated under appropriate conditions, and then tested for growth of the bacteria. Compounds with low or no antibiotic activity (a high MIC) will allow growth at high concentrations of compound, while compounds with high antibiotic activity will allow bacterial growth only at lower concentrations (a low MIC).

The assay uses stock bacterial culture conditions appropriate for the chosen strain of bacteria. Stock cultures from the permanent stock culture collection can be stored as frozen suspensions at −70° C. Cultures may be suspended in 10% skim milk (BD) prior to snap freezing in dry ice/ethanol and then placed in a −70° C. freezer. Cultures may be maintained on Tryptic Soy Agar containing 5% Sheep Blood at room temperature (20° C.), and each culture may be recovered from frozen form and transferred an additional time before MIC testing. Fresh plates are inoculated the day before testing, incubated overnight, and checked to confirm purity and identity.

The identity and purity of the cultures recovered from the stock culture can be confirmed to rule out the possibility of contamination. The identity of the strains may be confirmed by standard microbiological methods (See, e.g., Murray et al., Manual of Clinical Microbiology, Eighth Edition. ASM Press {ISBN 1-55581-255-4}). In general, cultures are streaked onto appropriate agar plates for visualization of purity, expected colony morphology, and hemolytic patterns. Gram stains can also be utilized. The identities are confirmed using a MicroScan WalkAway 40 SI Instrument (Dade Behring, West Sacramento, Calif.). This device utilizes an automated incubator, reader, and computer to assess for identification purposes the biochemical reactions carried out by each organism. The MicroScan WalkAway can also be used to determine a preliminary MIC, which may be confirmed using the method described below.

Frozen stock cultures may be used as the initial source of organisms for performing microbroth dilution minimum inhibition concentration (MIC) testing. Stock cultures are passed on their standard growth medium for at least 1 growth cycle (18-24 hours) prior to their use. Most bacteria may be prepared directly from agar plates in 10 mL aliquots of the appropriate broth medium. Bacterial cultures are adjusted to the opacity of a 0.5 McFarland Standard (optical density value of 0.28-0.33 on a Perkin-Elmer Lambda EZ150 Spectrophotometer, Wellesley, Mass., set at a wavelength of 600 nm). The adjusted cultures are then diluted 400 fold (0.25 mL inoculum+100 mL broth) in growth media to produce a starting suspension of approximately 5×105 colony forming units (CFU)/mL. Most bacterial strains may be tested in cation adjusted Mueller Hinton Broth (CAMHB).

Test compounds (“drugs”) are solubilized in a solvent suitable for the assay, such as DMSO. Drug stock solutions may be prepared on the day of testing. Microbroth dilution stock plates may be prepared in two dilution series, 64 to 0.06 μg drug/mL and 0.25 to 0.00025 μg drug/mL. For the high concentration series, 200 μL of stock solution (2 mg/mL) is added to duplicate rows of a 96-well microtiter plate. This is used as the first well in the dilution series. Serial two-fold decremental dilutions are made using a BioMek FX robot (Beckman Coulter Inc., Fullerton, Calif.) with 10 of the remaining 11 wells, each of which will contain 100 μL of the appropriate solvent/diluent. Row 12 contains solvent/diluent only and serves as the control. For the first well of the low concentration series, 200 μL of an 8 μg/mL stock are added to duplicate rows of a 96-well plate. Serial two-fold dilutions are made as described above.

Daughter 96-well plates may be spotted (3.2 μL/well) from the stock plates listed above using the BioMek FX robot and used immediately or frozen at −70° C. until use. Aerobic organisms are inoculated (100 μL volumes) into the thawed plates using the BioMek FX robot. The inoculated plates are be placed in stacks and covered with an empty plate. These plates are then incubated for 16 to 24 hours in ambient atmosphere according to CLSI guidelines (National Committee for Clinical Laboratory Standards, Methods for Dilution, Antimicrobial Tests for Bacteria that Grow Aerobically; Approved Standard-Sixth Edition. NCCLS document M7-A6 {ISBN 1-56238-486-4}).

After inoculation and incubation, the degree of bacterial growth can be estimated visually with the aid of a Test Reading Mirror (Dynex Technologies 220 16) in a darkened room with a single light shining directly through the top of the microbroth tray. The MIC is the lowest concentration of drug that prevents macroscopically visible growth under the conditions of the test.

### Fungus for Treatment/Induction of Cell Death

Exemplary fungus that may be treated include, for example, fungus from the genus Acremonium, Absidia, Alternaria, Aspergillus, Aureobasidium, Basidiobolus, Bjerkandera, Blastomyces, Candida, Cephalosporium, Ceriporiopsis, Chaetomium, Chrysosporium, Cladosporium, Coccidioides, Conidiobolus, Coprinus, Coriolus, Corynespora, Cryptococcus, Curvularia, Cunninghamella, Exophiala, Epidermophyton, Filibasidium, Fonsecaea, Fusarium, Geotrichum, Hendersonula, Histoplasma, Humicola, Leptosphaeria, Loboa, Madurella, Malassezia, Microsporum, Mycocentrospora, Mucor, Neotestudina, Paecilomyces, Paracoccidioides, Penicillium, Phialophora, Pneumocystis, Pseudallescheria, Rhinosporidium, Rhizomucor, Rhizopus, Saccharomyces, Scopulariopsis, Sporothrix, Talaromyces, Thermoascus, Thielavia, Tolypocladium, Trametes, Trichoderma, Trichophyton, Trichosporon, or Wangiella. In certain embodiments, the fungus is an Acremonium, Absidia (e.g., Absidia corymbifera), Alternaria, Aspergillus (e.g., Aspergillus clavatus, Aspergillus flavus, Aspergillus fumigatus, Aspergillus nidulans, Aspergillus niger, Aspergillus terreus, and Aspergillus versicolor), Aureobasidium, Basidiobolus, Blastomyces (e.g., Blastomyces dermatitidis), Candida (e.g., Candida albicans, Candida glabrata, Candida guilliermondii, Candida kefyr, Candida krusei, Candida lusitaniae, Candida parapsilosis, Candida pseudotropicalis, Candida stellatoidea, Candida tropicalis, Candida utilis, Candida lipolytica, Candida famata and Candida rugosa), Cephalosporium, Chaetomium, Chrysosporium, Cladosporium (e.g., Cladosporium carrionii and Cladosporium trichloides), Coccidioides (e.g., Coccidioides immitis), Conidiobolus, Coprinus, Corynespora, Cryptococcus (e.g., Cryptococcus neoformans), Curvularia, Cunninghamella (e.g., Cunninghamella elegans), Exophiala (e.g., Exophiala dermatitidis and Exophiala spinifera), Epidermophyton (e.g., Epidermophyton floccosum), Fonsecaea (e.g., Fonsecaea pedrosoi), Fusarium (e.g., Fusarium solani), Geotrichum (e.g., Geotrichum candiddum and Geotrichum clavatum), Hendersonula, Histoplasma, Leptosphaeria, Loboa, Madurella, Malassezia (e.g., Malassezia furfur), Microsporum (e.g., Microsporum canis and Microsporum gypseum), Mycocentrospora, Mucor, Neotestudina, Paecilomyces, Paracoccidioides (e.g., Paracoccidioides brasiliensis), Penicillium (e.g., Penicillium marneffei), Phialophora, Pneumocystis (e.g., Pneumocystis carinii), Pseudallescheria (e.g., Pseudallescheria boydii), Rhinosporidium, Rhizomucor, Rhizopus (e.g., Rhizopus microsporus var. rhizopodiformis and Rhizopus oryzae), Saccharomyces (e.g., Saccharomyces cerevisiae), Scopulariopsis, Sporothrix (e.g., Sporothrix schenckii), Trichophyton (e.g., Trichophyton mentagrophytes and Trichophyton rubrum), Trichosporon (e.g., Trichosporon asahii, Trichosporon beigelii and Trichosporon cutaneum), and Wangiella.

In certain other embodiments, the fungus is Aspergillus awamori, Aspergillus foetidus, Aspergillus funiigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearurn, Fusarium graminum, Fusarium heterosporum, Fusarium negimdi, Fusarium oxvsporum, Fusarium reticulatum, Fusarium roseum, Fusarium sambucinum, Fusarium sarcochrourn, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpiirogenum, Phanerochaete chrysosporium, Phlehia radiata, Pleurolus eryngii, Thielavia terrestris, Trametes villosa, Trametes versicolor, Trichoderma harzianum, Trichoderma koningii, Trichoderma longibrachiatiim, Trichoderma reesei, or Trichoderma viride. In certain embodiments, methods herein comprise treating an infection by one or more of Aspergillus awamori, Aspergillus foetidus, Aspergillus funiigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearurn, Fusarium graminum, Fusarium heterosporum, Fusarium negimdi, Fusarium oxvsporum, Fusarium reticulatum, Fusarium roseum, Fusarium sambucinum, Fusarium sarcochrourn, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpiirogenum, Phanerochaete chrysosporium, Phlehia radiata, Pleurolus eryngii, Thielavia terrestris, Trametes villosa, Trametes versicolor, Trichoderma harzianum, Trichoderma koningii, Trichoderma longibrachiatiim, Trichoderma reesei, or Trichoderma viride.

### Manufacture of Medicaments

Another aspect of the invention provides for the use of a deuterium-enriched compound described herein in the manufacture of a medicament. The medicament may be for treating one or more of the medical disorders described herein, such as treating a bacterial infection or fungal infection.

### III. Dosing Considerations and Combination Therapy

Doses of a compound provided herein, or a pharmaceutically acceptable salt thereof, vary depending on factors such as: specific indication to be treated; age and condition of a patient; and amount of second active agent used, if any. Generally, a compound provided herein, or a pharmaceutically acceptable salt thereof, may be used in an amount of from about 0.1 mg to about 1 g per day, or from about 0.1 mg to about 500 mg per day, and can be adjusted in a conventional fashion (e.g., the same amount administered each day of the treatment), in cycles (e.g., one week on, one week off), or in an amount that increases or decreases over the course of treatment. In other embodiments, the dose can be from about 1 mg to about 500 mg, from about 0.1 mg to about 150 mg, from about 1 mg to about 300 mg, from about 10 mg to about 100 mg, from about 0.1 mg to about 50 mg, from about 1 mg to about 50 mg, from about 10 mg to about 50 mg, from about 20 mg to about 30 mg, or from about 1 mg to about 20 mg.

In yet other embodiments, the daily dose can be from about 0.5 mg to 5 mg, 5 mg to 10 mg, 10 mg to 20 mg, 20 mg to 35 mg, 35 mg to 50 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, or 475 mg to 500 mg. In certain embodiments, the daily dosage is in the range of about 1 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, or 400 mg to 500 mg. In yet other embodiments, the daily dose is less than about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, or 450 mg. In yet other embodiments, the daily dose is less than about 125 mg, 150 mg, or 175 mg.

Unless indicated otherwise, compounds described herein may be administered using any medically accepted route of administration. For example, in certain embodiments, unless indicated otherwise, the compound is administered by oral administration, injection, or transdermal administration. In a preferred embodiment, the compound is administered orally.

In certain aspects, the therapeutic agents provided herein are cyclically administered to a patient. Cycling therapy involves the administration of an active agent for a period of time, followed by a rest (i.e., discontinuation of the administration) for a period of time, and repeating this sequential administration. Cycling therapy can reduce the development of resistance to one or more of the therapies. These regimens can avoid or reduce the side effects of one of the therapies, and/or improve the efficacy of the treatment.

Consequently, in another aspect, a compound provided herein is administered daily in a single or divided doses in a four to six week cycle with a rest period of about a week or two weeks. Cycling therapy further allows the frequency, number, and length of dosing cycles to be increased. Thus, another aspect encompasses the administration of a compound provided herein for more cycles than are typical when it is administered alone. In yet another aspect, a compound provided herein is administered for a greater number of cycles than would typically cause dose-limiting toxicity in a patient to whom a second active ingredient is not also being administered.

In another aspect, a compound provided herein is administered daily and continuously for three or four weeks at a dose of from about 0.1 mg to about 500 mg per day, followed by a rest of one or two weeks. In other embodiments, the dose can be from about 1 mg to about 500 mg, from about 0.1 mg to about 150 mg, from about 1 mg to about 300 mg, from about 10 mg to about 100 mg, from about 0.1 mg to about 50 mg, from about 1 mg to about 50 mg, from about 10 mg to about 50 mg, from about 20 mg to about 30 mg, or from about 1 mg to about 20 mg, followed by a rest.

In another aspect, a compound provided herein and a second active ingredient are administered orally or parenterally, with administration of the compound provided herein occurring prior to (e.g., about 30 to 60 minutes) the second active ingredient, during a cycle of four to six weeks. In certain embodiments, the compound and second active agent are administered as a single dosage or they are administered separately. In another aspect, the combination of a compound provided herein and a second active ingredient is administered by intravenous infusion over about 90 minutes every cycle.

Typically, the number of cycles during which the combination treatment is administered to a patient will be from about one to about 24 cycles, from about two to about 16 cycles, or from about three to about four cycles.

### Combination Therapy

A compound provided herein, or a pharmaceutically acceptable salt thereof, can be combined with other pharmacologically active compounds (“second active agents”) in methods and compositions provided herein. Certain combinations may work synergistically in the treatment of particular types of diseases or disorders, and conditions and symptoms associated with such diseases or disorders. A compound provided herein, or a pharmaceutically acceptable salt thereof, can also work to alleviate adverse effects associated with certain second active agents, and vice versa.

One or more second active ingredients or agents can be used in the methods and compositions provided herein. Second active agents can be large molecules (e.g., proteins) or small molecules (e.g., synthetic inorganic, organometallic, or organic molecules).

Administration of a compound provided herein, or a pharmaceutically acceptable salt thereof, and the second active agent(s) to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. One route of administration for compounds provided herein is oral. Routes of administration for the second active agents or ingredients are known to those of ordinary skill in the art. See, e.g., Physicians' Desk Reference (60^{th} Ed., 2006).

A. Exemplary Second Active Agents for Treating Bacterial Infections

Exemplary second active agents (or additional therapeutic agents) for treating bacterial infections include, for example, an aminoglycoside, carbacephem, carbapenem, cephalosporin (e.g., first generation, second generation, third generation, or fourth generation), a glycopeptide, lipopeptide, macrolide, monobactam, penicillin, polypeptide, quinolone, sulfonamide, tetracycline, oxazolidinone, rifamycin, and various unclassified antibiotics (e.g., chloramphenicol), each of which is described in more detail below.

Penicillins include those antibiotic drugs obtained from penicillium molds or produced synthetically, which are most active against Gram-positive bacteria and used in the treatment of various infections and diseases. Penicillin is one of the beta-lactam antibiotics, all of which possess a four-ring beta-lactam structure fused with a five-membered thiazolidine ring. These antibiotics are nontoxic and kill sensitive bacteria during their growth stage by the inhibition of biosynthesis of their cell wall mucopeptide. Penicillin antibiotics provide narrow spectrum bioactivity, moderate or intermediate spectrum bioactivity, and broad spectrum bioactivity. Without limitation, narrow spectrum penicillins include methicillin, dicloxacillin, flucloxacillin, oxacillin, nafcillin, or the like. Without limitation, moderate or intermediate spectrum penicillins include amoxicillin, ampicillin, or the like. Penicillins include, without limitation, ampicillin, azlocillin, carbenicillin, cloxacillin, dicloxacillin, flucloxacillin, mezlocillin, nafcillin, penicillin, piperacillin, and ticarcillin.

Aminoglycosides are a group of antibiotics that are effective against certain types of bacteria. They include amikacin, gentamicin, kanamycin, neomycin, netilmicin, paromomycin, streptomycin, tobramycin and apramycin. Aminoglycosides are useful primarily in infections involving aerobic, gram-negative bacteria, such as Pseudomonas, Acinetobacter, and Enterobacter. In addition, some mycobacteria, including the bacteria that cause tuberculosis, are susceptible to aminoglycosides.

Carbacephem is a class of antibiotic medication, specifically modified forms of cephalosporin. Without limitation, carbacephems include loracarbef, or the like.

Carbapenems are a class of beta-lactam antibiotics, which include, without limitation, imipenem (often given as part of imipenem/cilastatin), meropenem, ertapenem, faropenem, doripenem, panipenem/betamipron, and the like.

Cephalosporins are a class of beta-lactam antibiotics. Together with cephamycins they belong to a sub-group called cephems. First generation cephalosporins include, without limitation, cefadroxil, cefazolin, and cephalexin. Second generation cephalosporin typically have a greater gram negative spectrum while retaining some activity against gram positive cocci. Second generation cephalosporins include, for example, cefonicid, cefprozil, cefproxil, cefuroxime, cefuzonam, cefaclor, cefamandole, ceforanide, and cefotiam. Third generation cephalosporins typically have a broad spectrum of activity and further increased activity against gram-negative organisms. Without limitation, third generation cephalosporins include cefcapene, cefdaloxime, cefdinir, cefditoren, cefetamet, cefixime, cefmenoxime, cefodizime, cefoperazone, cefotaxime, cefpimizole, cefpodoxime, cefteram, ceftibuten, ceftiofur, ceftiolene, ceftizoxime, and ceftriaxone. Third generation cephalosporins with antipseudomonal activity include ceftazidime, cefpiramide, and cefsulodin. Oxacephems are also sometimes grouped with third-generation cephalosporins and include latamoxef and flomoxef. Fourth generation cephalosporins are extended-spectrum agents typically with similar activity against Gram positive organisms as first-generation cephalosporins. Exemplary fourth generation cephalosporins include cefclidine, cefepime, cefluprenam, cefoselis, cefozopran, cefpirome, and cefquinome. These cephems have progressed far enough to be named, but have not been assigned to a particular generation: ceftobiprole, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftioxide, ceftobiprole, ceftobiprole, and cefuracetime.

Glycopeptide antibiotics feature a glycosylated cyclic or polycyclic nonribosomal peptide. Exemplary glycopeptide antibiotics include vancomycin, teicoplanin, ramoplanin, and decaplanin.

Macrolides are a group of drugs (typically antibiotics) whose activity stems from the presence of a macrolide ring, a large lactone ring to which one or more deoxy sugars, usually cladinose and desosamine, are attached. The lactone ring can be either 14-, 15- or 16-membered. Common antibiotic macrolides include erythromycin, azithromycin, troleandomycin, clarithromycin, dirithromycin, and roxithromycin.

Monobactams are beta-lactam antibiotics wherein the beta-lactam ring is alone, and not fused to another ring (in contrast to most other beta-lactams, which have at least two rings). An example is aztreonam.

Polypeptide antibiotics include bacitracin, colistin, and polymyxin B.

Quinolones are another family of broad spectrum antibiotics. The parent of the group is nalidixic acid. Exemplary quinolone antibiotics include cinoxacin, flumequine, nalidixic acid, oxolinic acid, piromidic acid, pipemidic acid, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin mesilate, sparfloxacin, temafloxacin, tosufloxacin, clinafloxacin, gemifloxacin, moxifloxacin, gatifloxacin, sitafloxacin, and trovafloxacin.

Antibacterial sulfonamides (sometimes called simply sulfa drugs) are synthetic antimicrobial agents that contain the sulfonamide group. In bacteria, antibacterial sulfonamides act as competitive inhibitors of the enzyme dihydropteroate synthetase, DHPS. Several antibacterial sulfonamides include, for example, mafenide prontosil, sulfacetamide, sulfamethizole, sulfanilamide, sulfasalazine, sulflsoxazole, trimethoprim, and trimethoprim-sulfamethoxazole.

Tetracyclines are a group of broad-spectrum antibiotics named for their four (“tetra-”) hydrocarbon rings (“-cycl-”) derivation (“-ine”). Exemplary tetracyclines include tetracycline, chlortetracycline, oxytetracycline, demeclocycline, doxycycline, lymecycline, meclocycline, methacycline, minocycline, rolitetracycline, and tigecycline.

Oxazolidinones are a class of compounds containing 2-oxazolidone in their structures. Oxazolidinones are useful antibiotics. Some of the most important oxazolidinones are the last generation of antibiotics used against gram-positive bacterial strains. One example of an oxazolidinone is linezolid.

Rifamycins are a group antibiotics that are synthesized either naturally by the bacterium Amycolatopsis mediterranei, or artificially. Rifamycins are particularly effective against mycobacteria, and are therefore used to treat tuberculosis, leprosy, and mycobacterium avium complex (MAC) infections. The rifamycin antibiotic group includes, without limitation, rifampin.

Lipopeptide antibiotics includes peptides with attached lipids or a mixture of lipids and peptides such as the cyclic lipopeptide, daptomycin.

Other unclassified antibiotics include chloramphenicol, clindamycin, ethambutol, fosfomycin, furazolidone, isoniazid, metronidazole, mupirocin, nitrofurantoin, platensimycin, pyrazinamide, quinupristin/dalfopristin, spectinomycin, and telithromycin.

B. Exemplary Second Active Agents for Treating Fungal Infections

Exemplary second active agents (or additional therapeutic agents) for treating fungal infections include, for example, 2-phenylphenol; 8-hydroxyquinoline sulphate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide, flutolanil; flutriafol; folpet; fosetyl-A1; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesil); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulphocarb; methfuroxam; metiram; metominostrobin; metsulphovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulphonyl)amino]-butanamide; 1-(1-naphthalenyl-1H-pyrrole-2,5-dione; 2,3,5,6-tetrachloro-4-(methylsulphonyl)-pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-6-methoxy-3-pyridinyl)-cyclopropanecarboxamide; N-butyl-8-(1,1-dimethylethyl)-1-oxaspiro-[4.5]decane-3-amine; sodium tetrathiocarbonate; and copper salts and preparations, such as Bordeaux mixture; copper hydroxide; copper naphthenate; copper oxychloride; copper sulphate; cufraneb; copper oxide; mancopper; oxine-copper. Bactericides: bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### IV. Pharmaceutical Compositions

The invention provides pharmaceutical compositions comprising a deuterium-enriched compound described herein, such as a compound of Formula I or II, and a pharmaceutically acceptable carrier. In certain embodiments, the pharmaceutical compositions comprise a therapeutically-effective amount of a deuterium-enriched compound described herein, such as a compound of Formula I or II, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Pharmaceutical compositions can be used in the preparation of individual, single unit dosage forms. Pharmaceutical compositions and dosage forms provided herein comprise a compound provided herein, or a pharmaceutically acceptable salt thereof. Pharmaceutical compositions and dosage forms can further comprise one or more excipients. Additionally, pharmaceutical compositions and dosage forms provided herein can comprise one or more additional active ingredients. Examples of optional second, or additional, active ingredients are described above.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th ed., Mack Publishing, Easton Pa. (1990).

The suitability of a particular excipient may depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose or other mono- or disaccharides. As used herein, the term “lactose-free” means that the amount of lactose present, if any, is insufficient to substantially increase the degradation rate of an active ingredient. Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In another aspect, lactose-free dosage forms comprise active ingredients, microcrystalline cellulose, pre-gelatinized starch, and magnesium stearate.

Also provided are anhydrous pharmaceutical compositions and dosage forms comprising active ingredients. Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are, in another aspect, packaged using materials known to prevent exposure to water such that they can be included in suitable formulary kits. Examples of suitable packaging include, but are not limited to, hermetically sealed foils, plastics, dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In another aspect, dosage forms comprise a compound provided herein in an amount of from about 0.10 to about 500 mg. Examples of dosages include, but are not limited to, 0.1, 1, 2, 5, 7.5, 10, 12.5, 15, 17.5, 20, 25, 50, 100, 150, 200, 250, 300, 350, 400, 450, or 500 mg.

In another aspect, dosage forms comprise the second active ingredient in an amount of 0.5 to about 1000 mg, from about 5 to about 500 mg, from about 10 to about 350 mg, or from about 50 to about 200 mg. Of course, the specific amount of the second active agent will depend on the specific agent used, the diseases or disorders being treated or managed, and the amount(s) of a compound provided herein, and any optional additional active agents concurrently administered to the patient.

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In another aspect, the invention provides oral dosage forms that are tablets or capsules, in which case solid excipients are employed. In another aspect, the tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinylpyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in another aspect, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In another aspect, pharmaceutical compositions comprise from about 0.5 to about 15 weight percent of disintegrant, or from about 1 to about 5 weight percent of disintegrant. Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a Syloid® silica gel (AEROSIL200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In another aspect, the invention provides a solid oral dosage form comprising a compound provided herein, anhydrous lactose, microcrystalline cellulose, polyvinylpyrrolidone, stearic acid, colloidal anhydrous silica, and gelatin.

Active ingredients provided herein can also be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated in its entirety herein by reference. Such dosage forms can be used to provide slow or controlled-release of one or more active ingredients using, for example, hydroxypropyl methyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In another aspect, the invention provides single unit dosage forms suitable for oral administration such as, but not limited to, tablets, capsules, gel caps, and caplets that are adapted for controlled-release.

Parenteral dosage forms can be administered to patients by various routes including, but not limited to, subcutaneous, intravenous (including bolus injection), intramuscular, and intraarterial. Administration of a parenteral dosage form bypasses a patient's natural defenses against contaminants, and thus, in these aspects, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of a compound provided herein. See, e.g., U.S. Pat. No. 5,134,127, which is incorporated in its entirety herein by reference.

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In another aspect, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are nontoxic and pharmaceutically acceptable. Moisturizers or humectants can also be added to pharmaceutical compositions and dosage forms. Examples of additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Also, the polarity of a solvent carrier, its ionic strength, or tonicity can be adjusted to improve delivery. Compounds such as stearates can also be added to pharmaceutical compositions or dosage forms to alter the hydrophilicity or lipophilicity of one or more active ingredients so as to improve delivery. In other aspects, stearates can serve as a lipid vehicle for the formulation, as an emulsifying agent or surfactant, or as a delivery-enhancing or penetration-enhancing agent. In other aspects, salts of the active ingredients can be used to further adjust the properties of the resulting composition.

In another aspect, the active ingredients provided herein are not administered to a patient at the same time or by the same route of administration. In another aspect, provided are kits which can simplify the administration of appropriate amounts of active ingredients.

In another aspect, the invention provides a kit comprising a dosage form of a compound provided herein. Kits can further comprise additional active ingredients or a pharmacologically active mutant or derivative thereof, or a combination thereof. Examples of the additional active ingredients include, but are not limited to, those disclosed herein.

In other aspects, the kits can further comprise devices that are used to administer the active ingredients. Examples of such devices include, but are not limited to, syringes, drip bags, patches, and inhalers.

### V. Definitions

To facilitate an understanding of the present invention, a number of terms and phrases are defined below.

The term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

Unless indicated otherwise, when a D is specifically recited at a position or is shown in a formula, this D represents a mixture of hydrogen and deuterium where the amount of deuterium is about 100% (i.e., the abundance of deuterium ranges from greater than 90% up to 100%). In certain embodiments, the abundance of deuterium in D is from 95% to 100%, or from 97% to 100%.

The term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

“Therapeutically effective amount” includes an amount of a compound of the invention that is effective when administered alone or in combination to treat the desired condition or disorder. “Therapeutically effective amount” includes an amount of the combination of compounds claimed that is effective to treat the desired condition or disorder. The combination of compounds can be additive and is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower incidence of adverse side effects and/or toxicity, increased efficacy, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, bisulfuric, carbonic, citric, edetic, ethanedisulfonic, ethanesulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauric, lauryl sulfonic, maleic, malic, mandelic, methanesulfonic, napsylic, naphthylic, nitric, oleic, oxalic, palmitic, pamoic, pantothenic, phenylacetic, phosphoric, polygalacturonic, propionic, salicylic, stearic, succinic, sulfamic, sulfanilic, sulfuric, tannic, tartaric, toluenesulfonic, and valeric. (See, for example, Berge et al. (1977) “Pharmaceutical Salts”, J. Pharm. Sci. 66:1-19.). In certain embodiments, the pharmaceutically acceptable salt is a hydrochloric acid salt.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or a water/oil emulsion), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, if a variable is not accompanied by a definition, then the previous definition of the variable controls.

Finally, the invention may be embodied in other specific forms without departing from the spirit or essential attributes thereof. This invention encompasses all combinations of aspects and embodiments of the invention noted herein. It is understood that any and all aspects of the invention may be taken in conjunction with any other aspects and/or embodiments to describe additional aspects. It is also to be understood that each individual element of the aspects is intended to be taken individually as its own independent aspect. Furthermore, any element of an aspect is meant to be combined with any and all other elements from any aspect to describe an additional aspect.

### Examples

The invention now being generally described, will be more readily understood by reference to the following examples, which are included merely for purposes of illustration of certain aspects and embodiments of the present invention, and are not intended to limit the invention.

### Example 1—Preparation of Racemic Deuterated Pioglitazone, (Rac-5-({p-[2-(5-Ethyl-2-Pyridyl)Ethoxy]Phenyl}Methyl)-(5-²H)-1,3-Thiazolidine-2,4-Dione)

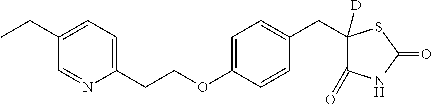 [SMILES: [2H]C1(Cc2ccc(OCCc3ccc(CC)cn3)cc2)SC(=O)NC1=O]

The hydrochloric acid salt of rac-5-({p-[2-(5-ethyl-2-pyridyl)ethoxy]phenyl}methyl)-1,3-thiazolidine-2,4-dione (i.e., pioglitazone hydrochloride) (1.5 g, 3.8 mmol) was placed in an oven-dried 250 mL round bottomed flask. Per-deuterated dimethylsulfoxide (d₆-DMSO, 18 mL) and triethylamine (1.596 mL, 11.5 mmol, 3 equiv.) were added, followed by per-deuterated methanol (d₄-MeOH, 14 mL). The resulting suspension was stirred at room temperature while monitoring by LC-MS. After 90 hours, d₆-DMSO (12 mL) and d₄-MeOH (16 mL) were added to dissolve the remaining solid. After another 18 hours (total 108 hours), LC-MS analysis showed almost complete deuterium incorporation with % D=98.3% at the chiral center. The mixture was concentrated under reduced pressure, then the concentrate was cooled to 0° C. and diluted with cold water (200 mL). The white solid that formed was filtered. The filtrate was extracted with ether (2×200 mL), and the organic layers were combined, dried over sodium sulfate (Na₂SO₄), and filtered. The white solid was combined with the filtrate. The solvent was evaporated under reduced pressure and the residue was dried overnight in vacuo to give 1.292 g (3.61 mmol) of rac-5-({p-[2-(5-ethyl-2-pyridyl)ethoxy]phenyl}methyl)-(5-²H)-1,3-thiazolidine-2,4-dione as a white solid. Overall yield: 1.292 g (3.61 mmol, 95%), % D=98% at the chiral center.

### Example 2—Isolation of Enantiopure (R)-Deuterated Pioglitazone and (S)-Deuterated Pioglitazone, ((5R)-5-({p-[2-(5-Ethyl-2-Pyridyl)Ethoxy]Phenyl}Methyl)-(5-²H)-1,3-Thiazolidine-2,4-Dione and (5S)-5-({p-[2-(5-Ethyl-2-Pyridyl)Ethoxy]Phenyl}Methyl)-(5-²H)-1,3-Thiazolidine-2,4-Dione)

rac-5-({p-[2-(5-Ethyl-2-pyridyl)ethoxy]phenyl}methyl)-(5-²H)-1,3-thiazolidine-2,4-dione (413 mg, 1.155 mmol) was dissolved in 30 mL of acetonitrile and 2-propanol (1:1 v/v). Trifluoroacetic acid (TFA, 225 μL) was added and the enantiomers (2 mL per run) were separated by supercritical fluid chromatography using a ChiralPak AD-H column (21×250 mm) and a mobile phase of 30% acetonitrile:2-propanol (1:1 v/v) in carbon dioxide (CO₂). Peaks were detected by their UV signal at 254 nm. Fractions containing each enantiomer were pooled and evaporated. Purity and enantiomeric excess (% ee) were determined by supercritical fluid chromatography using an analytical ChiralPak AD-H column (4.6×100 mm) and the same mobile phase. Overall yield was 405.3 mg (1.134 mmol, 98%). The absolute configuration of each enantiomer was determined by measurement of its specific rotation in dioxane and then comparison with specific rotation data already published for the enantiomers of pioglitazone and deuterated pioglitazone (see International Patent Application Publication Nos. WO 2010/015818 and WO 2010/150014). Physical characterization data for each enantiomer is provided below.

(S)-5-({p-[2-(5-Ethyl-2-pyridyl)ethoxy]phenyl}methyl)-(5-²H)-1,3-thiazolidine-2,4-dione (i.e., deuterated (S)-pioglitazone): 214.2 mg (0.599 mmol), 99.6% purity (UV, 220 nm), 99.0% ee: LC/MS: 358.26 (M+1) (>99% deuterium incorporation at the chiral center); ¹H NMR (300 MHz, d₆-DMSO) δ (ppm): 8.34 (s, 1H), 7.55 (d, 1H, J=7.8 Hz), 7.25 (d, 1H, J=7.8 Hz), 7.11 (d, 2H, J=8.7 Hz), 6.84 (d, 2H, J=8.7 Hz), 4.29 (t, 2H, J=6.6 Hz), 3.28 (d, 1H, J=13.2 Hz), 3.12 (t, 2H, J=6.6 Hz), 3.03 (d, 1H, J=14.4 Hz), 2.58 (q, 2H, J=7.7 Hz), 1.16 (t, 3H, J=7.5 Hz); specific rotation [α]_{D}=−72.4° (c 0.5, 19° C., dioxane).

(R)-5-({p-[2-(5-Ethyl-2-pyridyl)ethoxy]phenyl}methyl)-(5-²H)-1,3-thiazolidine-2,4-dione (i.e., deuterated (R)-pioglitazone): 191.1 mg (0.535 mmol), 100% purity (UV, 220 nm), 100% ee: LC/MS: 358.26 (M+1) (>99% deuterium incorporation at the chiral center); ¹H NMR (300 MHz, d₆-DMSO) δ (ppm): 8.34 (s, 1H), 7.55 (d, 1H, J=7.8 Hz), 7.25 (d, 1H, J=7.8 Hz), 7.11 (d, 2H, J=8.7 Hz), 6.84 (d, 2H, J=8.7 Hz), 4.29 (t, 2H, J=6.6 Hz), 3.28 (d, 1H, J=13.2 Hz), 3.12 (t, 2H, J=6.6 Hz), 3.03 (d, 1H, J=14.4 Hz), 2.58 (q, 2H, J=7.7 Hz), 1.16 (t, 3H, J=7.5 Hz); specific rotation [α]_{D}=+94.3 (c 0.5, 19° C., dioxane).

### Example 3—PPARγ Agonist Activity

Agonist activity of deuterated pioglitazone towards the peroxisome proliferator-activated receptor gamma (PPARγ) was evaluated in two separate experiments. Experimental procedures and results from the first experiment are provided in Part I below. Experimental procedures and results from the second experiment are provided in Part II below. A discussion of the results from each experiment are provided in Part III below.

### Part I—Analysis of PPARγ Agonist Activity of (S)-5-({p-[2-(5-Ethyl-2-pyridyl)ethoxy]phenyl}methyl)-(5-²H)-1,3-thiazolidine-2,4-dione (d-S-pio) and (R)-5-({p-[2-(5-Ethyl-2-pyridyl)ethoxy]phenyl}methyl)-(5-²H)-1,3-thiazolidine-2,4-dione (d-R-pio)

Agonist activity of the enantiomers of 5-({p-[2-(5-ethyl-2-pyridyl)ethoxy]phenyl}methyl)-(5-²H)-1,3-thiazolidine-2,4-dione at the peroxisome proliferator-activated receptor gamma (PPARγ) was evaluated in the thyroid receptor-associated protein complex, 220 kDa component (TRAP220) PPARγ coactivator recruitment assay performed at Cerep (France). Briefly, a mixture of labeled PPARγ and tagged TRAP220 coactivator was pre-incubated at room temperature for 30 minutes in the presence of a PPARγ-targeted fluorescence acceptor and test compound. A TRAP220-targeted fluorescence donor was then added and the mixture was incubated for 120 minutes at room temperature. Next, the fluorescence signal was measured and results expressed as a percent of control (10 μM rosiglitazone). A dose response curve was generated for each enantiomer and the experimental data was analyzed using the log(agonist) vs. response (three parameters) non-linear model in GraphPad Prism 6.0 (GraphPad Software, Inc., La Jolla, Calif.), with a fixed Hillslope of 1.

(S)-5-({p-[2-(5-Ethyl-2-pyridyl)ethoxy]phenyl}methyl)-(5-²H)-1,3-thiazolidine-2,4-dione (i.e., deuterated (S)-pioglitazone) was the most potent (EC₅₀=707 nM) and gave the highest maximum coactivator recruitment (106%). (R)-5-({p-[2-(5-Ethyl-2-pyridyl)ethoxy]phenyl}methyl)-(5-²H)-1,3-thiazolidine-2,4-dione (i.e., deuterated (R)-pioglitazone) was less potent (EC₅₀=4.4 μM) with only 29% maximum coactivator recruitment when compared to rosiglitazone.

### Part II—Analysis of PPARγ Agonist Activity of rac-5-({p-[2-(5-Ethyl-2-pyridyl)ethoxy]phenyl}methyl)-1,3-thiazolidine-2,4-dione (h-rac-pio); (S)-5-({p-[2-(5-Ethyl-2-pyridyl)ethoxy]phenyl}methyl)-(5-²H)-1,3-thiazolidine-2,4-dione (d-S-pio); and (R)-5-({p-[2-(5-Ethyl-2-pyridyl)ethoxy]phenyl}methyl)-(5-²H)-1,3-thiazolidine-2,4-dione (d-R-pio)

PPARγ agonist activity of the following compounds was evaluated in the thyroid receptor-associated protein complex, 220 kDa component (TRAP220) PPARγ coactivator recruitment assay performed at Cerep (France):
rac-5-({p-[2-(5-Ethyl-2-pyridyl)ethoxy]phenyl}methyl)-1,3-thiazolidine-2,4-dione
(S)-5-({p-[2-(5-Ethyl-2-pyridyl)ethoxy]phenyl}methyl)-(5-²H)-1,3-thiazolidine-2,4-dione
(R)-5-({p-[2-(5-Ethyl-2-pyridyl)ethoxy]phenyl}methyl)-(5-²H)-1,3-thiazolidine-2,4-dione.

The experimental procedure involved subjecting a mixture of labeled PPARγ and tagged TRAP220 coactivator to pre-incubation with a fluorescence acceptor at room temperature for 30 minutes in the presence of the test compound. A fluorescence donor was then added, and the mixture was incubated for 120 minutes at room temperature. Next, the fluorescence signal was measured and results expressed as a percent of control (10 μM rosiglitazone). A dose response curve was generated for each enantiomer and the experimental data was analyzed using the log(agonist) vs. response (three parameters) non-linear model in GraphPad Prism 6.0 (GraphPad Software, Inc., La Jolla, Calif.), with a fixed Hillslope of 1 and maximum of 100%.

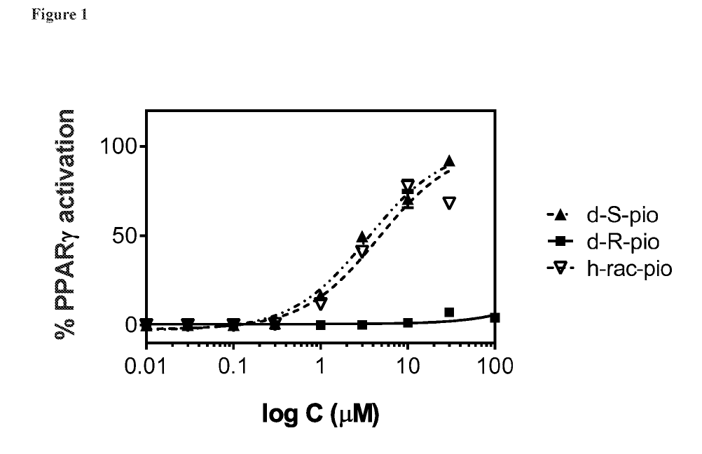

Experimental results are depicted in FIG. 1 and EC₅₀ values are provided in Table 5 below. d-S-pio was a more potent PPARγ agonist than h-rac-pio and d-R-pio. In this experiment, d-R-pio did not show any agonist activity at concentrations up to 100 μM.

TABLE 5

| Compound | EC₅₀ (μM) |
|---|---|
| d-S-pio | 3.47 |
| d-R-pio | >100 |
| h-rac-pio | 4.63 |

### Part III—Discussion of PPARγ Agonist Activity Results from Parts I and II

Experimental results in Parts I and II illustrate the trend that d-S-pio is a much more potent agonist of PPARγ than d-R-pio. Differences in the specific EC₅₀ values from the experiment in Parts I and II are understood to reflect typical differences observed in such cell-based assays between separate executions of the experiments. Such differences do not significantly impact characterization of the relative difference in PPARγ agonist activity for compounds tested under the same execution of the experiment.

### Example 4—Human and Mouse Plasma Stability Study of (S)-5-({P-[2-(5-Ethyl-2-Pyridyl)Ethoxy]Phenyl}Methyl)-1,3-Thiazolidine-2,4-Dione; (R)-5-({P-[2-(5-Ethyl-2-Pyridyl)Ethoxy]Phenyl}Methyl)-1,3-Thiazolidine-2,4-Dione; and Rac-5-({P-[2-(5-Ethyl-2-Pyridyl)Ethoxy]Phenyl}Methyl)-(5-²H)-1,3-Thiazolidine-2,4-Dione

rac-5-({p-[2-(5-Ethyl-2-pyridyl)ethoxy]phenyl}methyl)-(5-²H)-1,3-thiazolidine-2,4-dione (d-rac-pio; a 1:1 mixture of (S)- and (R)-enantiomers 5-({p-[2-(5-ethyl-2-pyridyl)ethoxy]phenyl}methyl)-(5-²H)-1,3-thiazolidine-2,4-dione) (d-rac-pio), (R)-5-({p-[2-(5-ethyl-2-pyridyl)ethoxy]phenyl}methyl)-1,3-thiazolidine-2,4-dione (h-R-pio), and (S)-5-({p-[2-(5-ethyl-2-pyridyl)ethoxy]phenyl}methyl)-1,3-thiazolidine-2,4-dione (h-S-pio) were dissolved in separate solutions of dimethylsulfoxide (DMSO). The stock solutions were diluted 1:49 v/v in C57BL/6 mouse or human plasma to 10, 5, and 5 μM concentrations for d-rac-pio, h-S-pio, and h-R-pio respectively. The plasma samples were incubated at 37° C. in duplicate (anticoagulant: K3EDTA). Aliquots (20 μL) were removed at t=0, 0.25, 0.5, 1, 2, 4, 8, 16, and 32 h, added to 130 μL acetonitrile, and vortexed. Samples were placed at −80° C. until the study was complete. After thawing, a 1:1 acetonitrile:water solution of internal standard (ISTD, d₄-pioglitazone, 1.6 μM) was added. The vortexed samples were centrifuged and 50 μL of supernatant was dispensed in a 96-well plate. These were further diluted with 200 μL of 0.1% acetic acid in water:acetonitrile 15:85 v/v.

The samples were analyzed semi-quantitatively by LC/MS-MS in MRM (multiple reaction monitoring) mode for concentrations of h-S-pio, h-R-pio, d-S-pio, and d-R-pio using a chiral column (ChiralPak IE-3, Chiral Technologies, West Chester, Pa.) for the separation of enantiomers (isocratic eluent: 01.% acetic acid in water/acetonitrile 15/85 v/v at 1 mL/min). All peak areas were normalized to the ISTD and peak areas for the deuterated enantiomers, d-S-pio and d-R-pio, were corrected for the isotopic peak of the corresponding protonated enantiomer, if present (an interference of 12.2% of the response for the protonated compound was determined experimentally). Corrected data were analyzed and plotted using Microsoft Excel 2013 (Microsoft Corp, Redmond, Wash.) and the Excel Solver as well as GraphPad Prism 6.0 (GraphPad Software LLC, La Jolla, Calif.) where appropriate.

Scheme 3 illustrates the possible reactions in a solution of deuterated racemic pioglitazone, where the abbreviations d-S, d-R, h-S, and h-R represent d-S-pio, d-R-pio, h-S-pio, and h-R-pio, respectively. The deuterium in both enantiomers, d-S and d-R, can be lost by D/H exchange to give both protonated enantiomers, h-S and h-R with rate constants k_{DRR}, k_{DRS}, k_{DSR}, and k_{DSS}. At the same time, the protonated enantiomers h-S and h-R can exchange with enantiomerization rate constants k_{RS} and k_{SR}. All four compounds can also degrade with potentially different degradation rate constants k_{dSd}, k_{dRd}, k_{hSd}, and k_{hRd}.

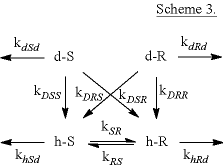

Human and mouse plasma data were analyzed independently. In addition, since the sum of peak areas for all enantiomeric isotopomers (h-S-pio+h-R-pio+d-S-pio+d-R-pio) appeared independent of incubation time in plasma from both species, degradation was considered negligible. Therefore, degradation rate constants k_{hSd}, k_{hRd}, h_{dSd}, and k_{dRd} were set to 0. Data in each species was analyzed using a stepwise approach. Data for the enantiomerization reaction of h-S-pio and h-R-pio were fitted first and independently of each other. The average rate constants, k_{SR} and k_{RS}, obtained from these analyses were used and fixed in the fitting of the stability data of d-rac-pio. Rate constants k_{DSS}, k_{DSR}, k_{DRS}, and k_{DRR} were obtained from this final analysis. Half-lives for the 4 enantiomeric isotopomers were then calculated as t_{1/2}=ln(2)/k, where k=k_{DSR}+k_{DSS} or k_{DRR}+k_{DRS} for the deuterated enantiomers (d-S-pio and d-R-pio, respectively) and k=k_{SR} or k_{RS} for the protonated enantiomers (h-S-pio and h-R-pio, respectively).

Data analysis was performed in Microsoft Excel 2013, using the Solver Generalized Reduced Gradient Nonlinear method with central derivatives to minimize the sum of sums of weighted Δ², square of difference between ISTD-normalized experimental data and calculated value, divided by the experimental data (both protonated enantiomers or both protonated and deuterated enantiomers). Calculated concentrations were obtained through numerical approximation of differential equations (1) and (2) for the stability studies of h-S-pio and h-R-pio, and equations (3) to (6) for the stability study of d-rac-pio by the Euler method (equation (7)). The step between calculated time points was minimized in order to minimize the local error (proportional to the square of the step size) and the global error (proportional to the step size).

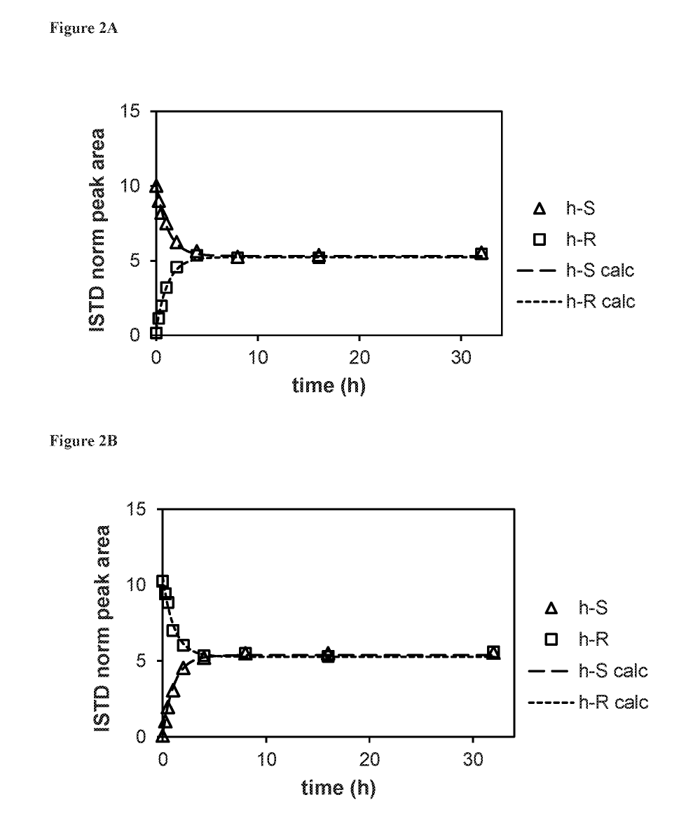

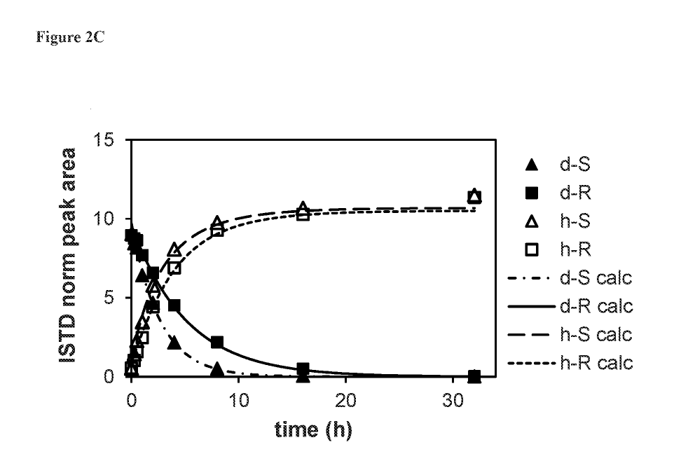

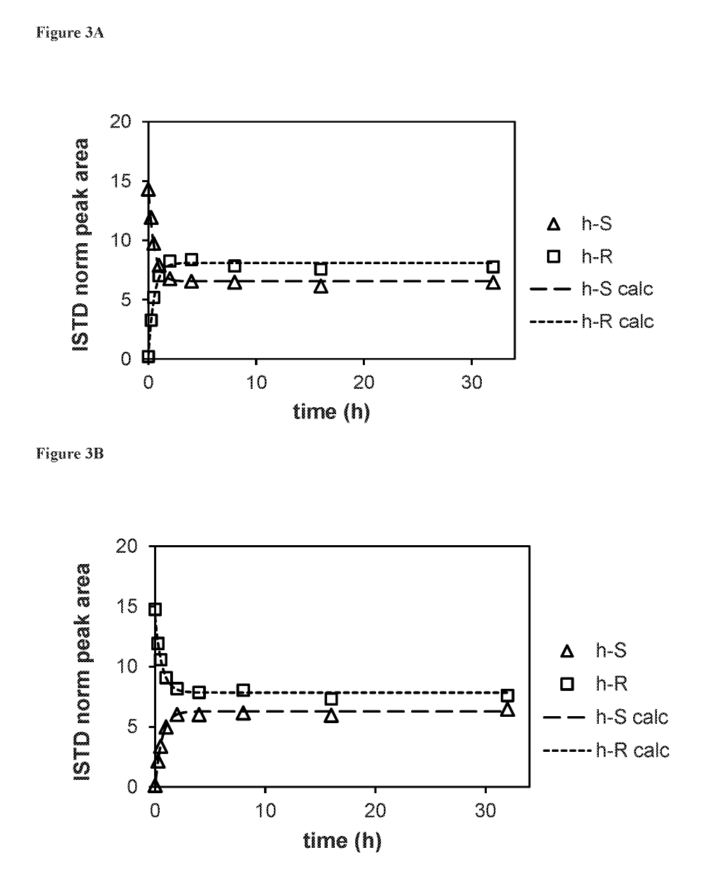

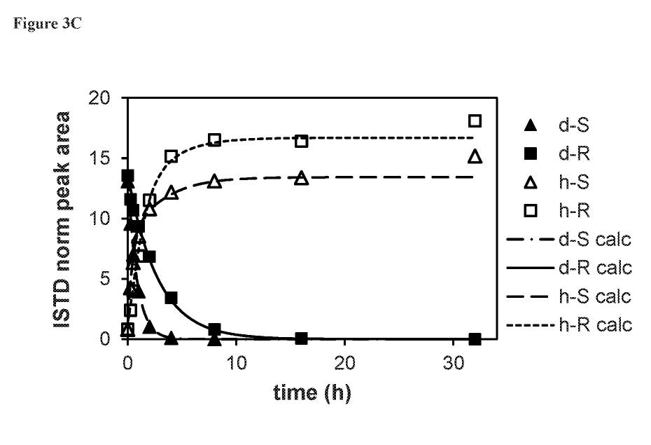

The observed and fitted data are shown in FIGS. 2A-C for stability in human plasma. The observed and fitted data are shown in FIGS. 3A-C for stability in mouse plasma. Fitted parameters are presented in Table 6, which provides rate constants and calculated half-lives (t_{1/2}) for the in vitro stability of h-S-pio, h-R-pio, and d-rac-pio in human and mouse plasma at 37° C. obtained by fitting experimental data to equations 1 to 6; the DXY stand for the D/H exchange reactions from d-S-pio (X═S) or d-R-pio (X═R) to h-S-pio (Y═S) or h-R-pio (Y═R); SR and RS represent the enantiomerization reaction h-S-pio to h-R-pio and h-R-pio to h-S-pio, respectively.

$$\frac{d [ h - S ]}{dt} = - {k}_{SR} [ h - S ] + {k}_{RS} [ h - R ] \\ \frac{d [ h - R ]}{dt} = {k}_{SR} [ h - S ] - {k}_{RS} [ h - R ] \\ \frac{d [ h - S ]}{dt} = - {k}_{SR} [ h - S ] + {k}_{RS} [ h - R ] + {k}_{DSS} [ d - S ] + {k}_{DRS} [ d - R ] \\ \frac{d [ h - R ]}{dt} = {k}_{SR} [ h - S ] - {k}_{RS} [ h - R ] + {k}_{DSR} [ d - S ] + {k}_{DRR} [ d - R ] \\ \frac{d [ d - S ]}{dt} = - ( {k}_{DSS} + {k}_{DSR} ) [ d - S ] \\ \frac{d [ d - R ]}{dt} = - ( {k}_{DRR} + {k}_{DRS} ) [ d - R ] \qquad Equations 1 - 6$$

where [h−S], [h−R], [d−S], [d−R] are the concentrations of h-S-pio, h-R-pio, d-S-pio, and d-R-pio, respectively; k_{SR} and k_{RS} are the rate constants for the enantiomerization reactions h-S-pio to h-R-pio and h-R-pio to h-S-pio, respectively; k_{DRR}, k_{DRS}, k_{DSR}, and k_{DSS} are the rate constants for the D/H exchange reactions d-S-pio or d-R-pio to h-S-pio or h-R-pio.

[X]ₜ₂=[X]ₜ₁+(t₂−t₁)[d[X]]ₜ₁ Equation 7

where [X]ₜᵢ is the concentration of h-S-pio, h-R-pio, d-S-pio or d-R-pio at time ti (wherein i=1 or 2, i.e., ti=t1 or t2), t1 is a time at which [X] is known, t2 is a time at which [X] is calculated, and [d[X]]ₜ₁ is the calculated value of the differential equation at time t1.

The equilibrium ratio of enantiomers h-R/h-S was approximately 1:1 in human plasma. The equilibrium ratio of enantiomers h-R/h-S was approximately 1.25:1 in mouse plasma. The effect of deuterium incorporation was different for the two enantiomers. For example, an approximately two-fold increased half-life was observed for d-R-pio vs. h-R-pio. However, the half-life of d-S-pio was approximately the same as the half-life for h-S-pio.

### Example 5—Monoamine Oxidase B (Mao-B) Inhibition Study of (S)-5-({P-[2-(5-Ethyl-2-Pyridyl)Ethoxy]Phenyl}Methyl)-(5-²H)-1,3-Thiazolidine-2,4-Dione (d-S-pio) and (R)-5-({P-[2-(5-Ethyl-2-Pyridyl)Ethoxy]Phenyl}Methyl)-(5-²H)-1,3-Thiazolidine-2,4-Dione (d-R-pio)

Separate dimethylsulfoxide (DMSO) stock solutions of d-S-pio and d-R-pio were serially diluted in DMSO then mixed with a solution containing 2.5 mU of human recombinant monoamine oxidase B in 90 mM 4-(2-hydroxyethyl)-1-piperazineethanesulfonic acid (HEPES) buffer containing 4.5% glycerol and 9% DMSO. The mixtures were incubated at 22° C. for 5 min. Substrate (D-luciferin derivative) was added, and then the mixture was incubated at 37° C. for 60 min. The reaction was stopped by addition of the detection reagent containing luciferase. Luminescence was read after standing for 60 min at room temperature.

Experiments were performed in duplicate and a positive control (deprenyl) was used to confirm the validity of the assay. Results were expressed as a percentage of the luminescence of the control (enzyme+substrate). IC₅₀ values were obtained by fitting experimental data (mean % luminescence as function of concentration) to the Hill equation with variable slope using non-linear regression analysis.

A greater than 5-fold difference in inhibition efficacy was observed between d-R-pio and d-S-pio. The results showed d-S-pio to have an IC₅₀=7.6 μM. The results showed d-R-pio to have an IC₅₀=1.4 μM.

### Example 6—Effect of Rac-5-({P-[2-(5-Ethyl-2-Pyridyl)Ethoxy]Phenyl}Methyl)-1,3-Thiazolidine-2,4-Dione (h-rac-pio); (S)-5-({P-[2-(5-Ethyl-2-Pyridyl)Ethoxy]Phenyl}Methyl)-(5-²H)-1,3-Thiazolidine-2,4-Dione (d-S-pio); and (R)-5-({P-[2-(5-Ethyl-2-Pyridyl)Ethoxy]Phenyl}Methyl)-(5-²H)-1,3-Thiazolidine-2,4-Dione (d-R-pio) on Mitochondrial Respiration in C2C12 Cells

The effect of treatment of intact C2C12 cells with h-rac-pio, d-S-pio, and d-R-pio on respiration was evaluated under mitochondrial stress conditions in the presence of sodium pyruvate, in an experimental setup similar to that reported by Divakurani et al. in Proc. Natl. Acad. Sci. 110 (2013), 5422-5427. Rosiglitazone (10 and 30 μM) and the mitochondrial pyruvate carrier inhibitor UK-5099 (300 nM) were used as positive controls.

On the day before the assay, C2C12 cells were subcultured in XF96 microplates at a density of 20,000 cells per well. After overnight incubation (37° C., 5% CO₂), the cells were washed 3 times with assay medium (Seahorse medium (Seahorse Bioscience, North Billerica, Mass.) containing 10 mM sodium pyruvate, pH 7.4). Compounds, d-S-pio, d-R-pio, or h-rac-pio (each at 3, 10, or 30 μM final concentration), were added to the wells and the plates were incubated at 37° C. (without CO₂). A full mitochondrial stress test was performed on the XF96 Extracellular Flux Analyzer (Seahorse Biosciences), including injection of the ATP synthase inhibitor oligomycin (3.5 μM final), the oxidative phosphorylation uncoupling agent FCCP (carbonyl cyanide 4-(trifluoromethoxy)phenylhydrazone, 1 μM final concentration), and the complex III inhibitor antimycin A (2.5 μM final). Incubation times of 15, 30, or 90 min were used prior to measurement of maximal respiration, i.e., until addition of FCCP. Injection of FCCP always occurred 15 min after the start of the respirometry assay. Maximal respiration as oxygen consumption rates (pmoles O₂/min) was measured for each compound, at each concentration, and each time point in three separate experiments.

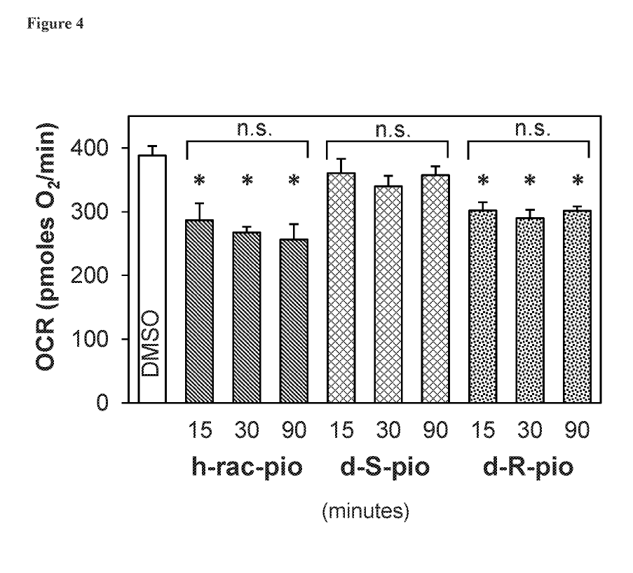

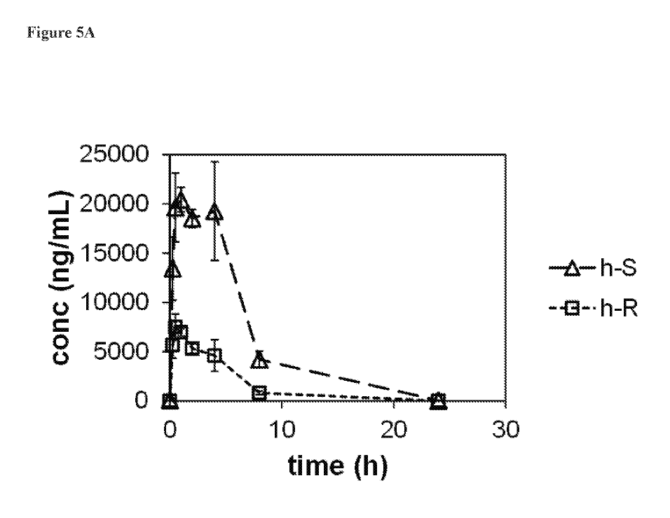

Results for the incubation with compounds at 30 μM concentration are presented in FIG. 4, which shows maximal respiration as oxygen consumption rate (OCR in pmoles O₂/min) of C2C12 cells treated with h-rac-pio, d-S-pio, or d-R-pio at 30 μM for 15, 30, or 90 minutes compared to the OCR in vehicle-treated cells (average of all repeats and time points); statistical analysis: one-way ANOVA with Newman-Keuls post-test; * P<0.05; n.s. means not statistically significant. Both h-rac-pio and d-R-pio inhibited maximal respiration while no significant measurable effect was observed for d-S-pio.

### Example 7—Pharmacokinetics (PK) of Rac-5-({P-[2-(5-Ethyl-2-Pyridyl)Ethoxy]Phenyl}Methyl)-1,3-Thiazolidine-2,4-Dione (h-Rac-Pio); (R)-5-({P-[2-(5-Ethyl-2-Pyridyl)Ethoxy]Phenyl}Methyl)-(5-²H)-1,3-Thiazolidine-2,4-Dione (d-R-pio); and (S)-5-({P-[2-(5-Ethyl-2-Pyridyl)Ethoxy]Phenyl}Methyl)-(5-²H)-1,3-Thiazolidine-2,4-Dione (d-S-pio)

### Part I—Experimental Procedure

Male C57BL/6 mice (8-10 weeks of age) were randomly divided into 3 groups of n=24 animals and administered 30 mg/kg h-rac-pio, 15 mg/kg d-S-pio, or 15 mg/kg d-R-pio (in a 0.25% carboxymethylcellulose solution prepared daily and used within 1 h of preparation) by oral gavage once a day for 5 days. Blood samples (˜0.5 mL) were collected in K2EDTA tubes on day 5 from n=3 animals per group per time point: pre-dose or 0.25, 0.5, 1, 2, 4, 8, or 24 h post-dose by retrobulbar bleeding under light anesthesia (isoflurane). Animals were then euthanized. Plasma was separated by centrifugation and stored at −80° C. until analyzed.

Samples were processed and analyzed by chiral HPLC/MS-MS (ISTD: d₄-pioglitazone) as described in Example 4. Peak areas were normalized to the peak area of the ISTD and normalized peak areas for deuterated enantiomers d-S-pio and d-R-pio were corrected for interference from the isotopic peak of the corresponding protonated enantiomer.

Concentrations were calculated by interpolation on standard curves generated from mouse plasma samples spiked with known concentrations of the pure analytes. Data was plotted in Excel 2013 (Microsoft Corp, Redmond, Wash.) and analyzed within Excel using the PKSolver add-in (version 2.0, as described in Zhang Y. et al. in Comput. Methods Programs Biomed. 99 (2010), 306-314) to determine PK parameters including exposure (as area under the curve, AUC) and elimination half-life (t_{1/2}).

### Part II—Results

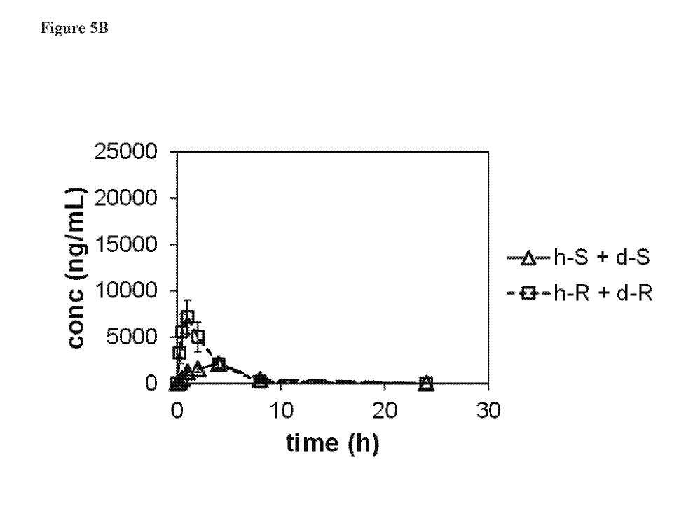

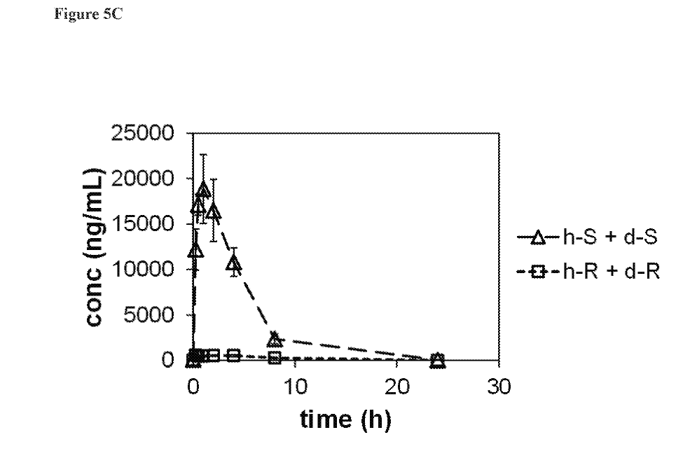

PK profiles determined using the above procedure are shown in FIGS. 5A-C. Selected PK parameters (Cₘₐₓ, tₘₐₓ, AUC_{0-inf}, and t_{1/2}) for the enantiomers of protonated and deuterated pioglitazone in mice after oral gavage of h-rac-pio, d-S-pio, or d-R-pio are presented in Table 7.

Exposure (as area under the curve, AUC) to the enantiomers of h-rac-pio was stereoselective in animals dosed with h-rac-pio (1:1 mixture of h-S-pio and h-R-pio) resulting in a 4:1 ratio of h-S-pio to h-R-pio. Dosing d-R-pio resulted in a 10-fold decrease in exposure to the (S)-enantiomer, while exposure to the (R)-enantiomer decreased by only 1.7-fold. Dosing d-R-pio resulted in a reversal of the relative exposure (S:R=3:5) compared to what was observed in mice dosed with h-rac-pio. Similarly, dosing d-S-pio resulted in a 7-fold decrease in exposure to the (R)-enantiomer (vs. dosing h-rac-pio) while exposure to the (S)-enantiomer decreased by 1.5-fold.

### INCORPORATION BY REFERENCE

All references listed herein are individually incorporated in their entirety by reference.

### EQUIVALENTS

Numerous modifications and variations of the invention are possible in light of the above teachings. It is therefore to be understood that within the scope of the appended claims, the invention may be practiced otherwise that as specifically described herein.

1. A method of treating an infection selected from the group consisting of a bacterial infection and a fungal infection, comprising administering to a patient in need thereof a therapeutically effective amount of a deuterium-enriched compound of Formula I having an optical purity of at least 75% enantiomeric excess to treat the infection, wherein Formula I is represented by: 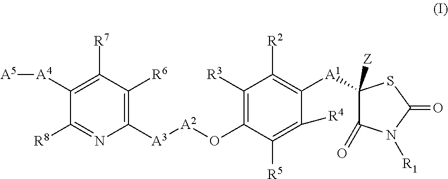
or a pharmaceutically acceptable salt thereof, wherein:
A¹, A², A³, and A⁴ are independently —C(R⁹)(R¹⁰)—;
A⁵ is —C(R¹¹)(R¹²)(R¹³);
R¹, R², R³, R⁴, R⁵, R⁶, R⁷, and R⁸ are independently H or D;
R⁹, R¹⁰, R¹¹, R¹², and R¹³ each represent independently for each occurrence H or D; and
Z is H or D, provided that the abundance of deuterium in Z is at least 30%.

2-26. (canceled)

27. The method of claim 1, wherein the compound is: 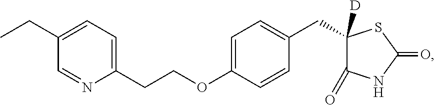 [SMILES: [2H][C@@]1(Cc2ccc(OCCc3ccc(CC)cn3)cc2)SC(=O)NC1=O]
or a pharmaceutically acceptable salt thereof, each having an optical purity of at least 90% enantiomeric excess.

28-32. (canceled)

33. A method of treating an infection selected from the group consisting of a bacterial infection and a fungal infection, comprising administering to a patient in need thereof a therapeutically effective amount of a deuterium-enriched compound of Formula II having an optical purity of at least 75% enantiomeric excess to treat the infection, wherein Formula II is represented by: 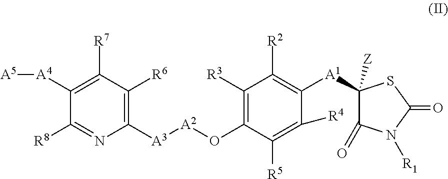
or a pharmaceutically acceptable salt thereof, wherein:
A¹, A², A³, and A⁴ are independently —C(R⁹)(R¹⁰)—;
A⁵ is —C(R¹¹)(R¹²)(R¹³);
R¹, R², R³, R⁴, R⁵, R⁶, R⁷, and R⁸ are independently H or D;
R⁹, R¹⁰, R¹¹, R¹², and R¹³ each represent independently for each occurrence H or D; and
Z is H or D, provided that the abundance of deuterium in Z is at least 30%.

34. The method of claim 33, wherein the infection is a bacterial infection.

35. The method of claim 34, wherein the bacterial infection comprises gram-positive bacteria.

36. The method of claim 34, wherein the bacterial infection comprises gram-negative bacteria.

37. (canceled)

38. (canceled)

39. The method of claim 34, wherein the bacterial infection is an infection by one or more of a Streptococccus, Escherichia, Klebsiella, Acinetobacter, Actinomyces, Anaerobiospirillum, Bacillus, Bacteroides, Bilophila, Campylobacter, Clostridium, Enterococcus, Eubacterium, Francisella, Fusobacterium, Haemophilus, Listeria, Moraxella, Mycobacterium, Neisseria, Peptostreptococci, Porphyromonas, Prevotella, Proteus, Pseudomonas, Salmonella, or Yersinia.

40. The method of claim 34, wherein the bacterial infection is an infection by one or more Streptococccus species, Escherichia species, Klebsiella species, Actinomyces species, Enterococcus species, Mycobacterium species, Neisseria species, or Pseudomonas species.

41. The method of claim 34, wherein the bacterial infection is an infection by one or more of Streptococcus pneumoniae, Streptococcus pyogenes, Staphylococcus aureus, Staphylococcus epidermidis, Acinetobacter baumannii, Bacillus anthracis, Bacteroides fragilis, Clostridium perfringens, Clostridium difficile, Escherichia coli, Enterococcus faecalis, Enterococcus faecium, Eubacterium lentum, Francisella tularensis, Fusobacterium nucleatum, Haemophilus influenzae, Klebsiella pneumoniae, Moraxella catarrhalis, Mycobacterium smegmatis, Mycobacterium tuberculosis, Neisseria gonorrhoeae, Porphyromonas asaccharolyticus, Proteus mirabilis, Pseudomonas aeruginosa, Salmonella typhimurium, or Yersinia enterocolytica.

42. The method of claim 34, wherein the bacterial infection is an infection by Streptococccus pneumoniae, Escherichia coli, or Klebsiella pneumoniae.

43. The method of claim 33, wherein the infection is a fungal infection.

44. The method of claim 43, wherein the fungal infection is an infection by one or more of an Acremonium, Absidia, Alternaria, Aspergillus, Aureobasidium, Basidiobolus, Bjerkandera, Blastomyces, Candida, Cephalosporium, Ceriporiopsis, Chaetomium, Chrysosporium, Cladosporium, Coccidioides, Conidiobolus, Coprinus, Coriolus, Corynespora, Cryptococcus, Curvularia, Cunninghamella, Exophiala, Epidermophyton, Filibasidium, Fonsecaea, Fusarium, Geotrichum, Hendersonula, Histoplasma, Humicola, Leptosphaeria, Loboa, Madurella, Malassezia, Microsporum, Mycocentrospora, Mucor, Neotestudina, Paecilomyces, Paracoccidioides, Penicillium, Phialophora, Pneumocystis, Pseudallescheria, Rhinosporidium, Rhizomucor, Rhizopus, Saccharomyces, Scopulariopsis, Sporothrix, Talaromyces, Thermoascus, Thielavia, Tolypocladium, Trametes, Trichoderma, Trichophyton, Trichosporon, or Wangiella.

45. The method of claim 43, wherein the fungal infection is an infection by one or more of Aspergillus awamori, Aspergillus foetidus, Aspergillus funiigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearurn, Fusarium graminum, Fusarium heterosporum, Fusarium negimdi, Fusarium oxvsporum, Fusarium reticulatum, Fusarium roseum, Fusarium sambucinum, Fusarium sarcochrourn, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpiirogenum, Phanerochaete chrysosporium, Phlehia radiata, Pleurolus eryngii, Thielavia terrestris, Trametes villosa, Trametes versicolor, Trichoderma harzianum, Trichoderma koningii, Trichoderma longibrachiatiim, Trichoderma reesei, or Trichoderma viride.

46. (canceled)

47. (canceled)

48. (canceled)

49. (canceled)

50. The method of claim 33, wherein the compound is a compound of Formula II-A having an optical purity of at least 75% enantiomeric excess, wherein Formula II-A is represented by: 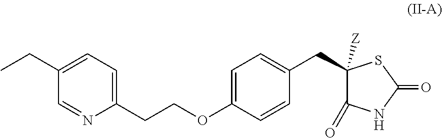
or a pharmaceutically acceptable salt thereof, wherein Z is H or D, provided that the abundance of deuterium in Z is at least 30%.

51. (canceled)

52. (canceled)

53. (canceled)

54. (canceled)

55. The method of claim 34, wherein the abundance of deuterium in Z is at least 90%.

56. (canceled)

57. (canceled)

58. (canceled)

59. The method of claim 34, wherein the compound is: 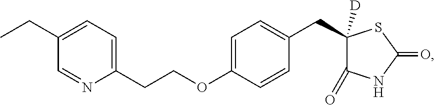 [SMILES: [2H][C@]1(Cc2ccc(OCCc3ccc(CC)cn3)cc2)SC(=O)NC1=O]
or pharmaceutically acceptable salt thereof, each having an optical purity of at least 90% enantiomeric excess.

60. The method of claim 34, wherein the compound is: 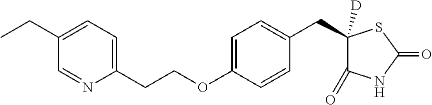 [SMILES: [2H][C@]1(Cc2ccc(OCCc3ccc(CC)cn3)cc2)SC(=O)NC1=O]
having an optical purity of at least 90% enantiomeric excess.

61. The method of claim 34, wherein the compound is: 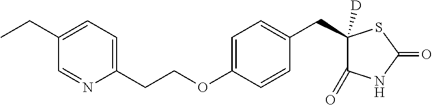 [SMILES: [2H][C@]1(Cc2ccc(OCCc3ccc(CC)cn3)cc2)SC(=O)NC1=O]
hydrochloride having an optical purity of at least 90% enantiomeric excess.

62. The method of claim 34, wherein the compound is: 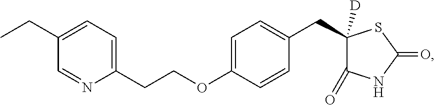 [SMILES: [2H][C@]1(Cc2ccc(OCCc3ccc(CC)cn3)cc2)SC(=O)NC1=O]
or pharmaceutically acceptable salt thereof, each having an optical purity of at least 95% enantiomeric excess.

63. The method of claim 34, wherein the compound is: 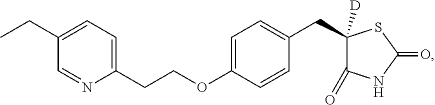 [SMILES: [2H][C@]1(Cc2ccc(OCCc3ccc(CC)cn3)cc2)SC(=O)NC1=O]
having an optical purity of at least 95% enantiomeric excess.

64. The method of claim 34, wherein the compound is: 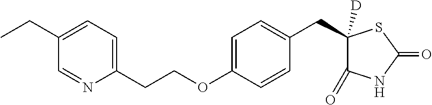 [SMILES: [2H][C@]1(Cc2ccc(OCCc3ccc(CC)cn3)cc2)SC(=O)NC1=O]
hydrochloride having an optical purity of at least 90% enantiomeric excess.
